(19) United States
(12) Patent Application Publication
(10) Pub. No.: US 2021/0393201 A1
(43) Pub. Date: Dec. 23, 2021

(54) Devices and Methods For Application Of Microneedle Arrays Using Radial And Axial Accelerations

(71) Applicant: Biolinq, Inc., San Diego, CA (US)

(72) Inventors: David Michael Morelock, Escondido, CA (US); Scott James Sylliaasen, San Diego, CA (US); George Albert Mansfield, III, San Diego, CA (US); Pradnya Prakash Samant, San Diego, CA (US); Buddhadev Paul Chaudhuri, Walnut Creek, CA (US); Andre Gamelin, Vista, CA (US); Jared Rylan Tangney, Encinitas, CA (US); Joshua Windmiller, San Diego, CA (US)

(73) Assignee: Biolinq, Inc., San Diego, CA (US)

(21) Appl. No.: 17/348,651

(22) Filed: Jun. 15, 2021

Related U.S. Application Data
(60) Provisional application No. 63/040,295, filed on Jun. 17, 2020.

(51) Int. Cl. A61B 5/00; A61B 5/145; A61B 5/1473; A61B 5/1459

(52) U.S. Cl. CPC A61B 5/685; A61B 5/1459; A61B 5/1473; A61B 5/14546

(57) ABSTRACT

A device and method for the insertion of an analyte-selective microneedle array sensor into a dermal stratum of a user is disclosed herein. The device comprises a body portion, a recessed actuation portion, a carrier, a gating feature, and a disengagement feature. A user-directed application of a specified force to the actuation area causes the carrier to overcome the gating feature, thereby to effect the acceleration of the microneedle array sensor device towards the skin surface of a user with a specified impact force and velocity.

### CROSS REFERENCE TO RELATED APPLICATION

The Present application claims priority to U.S. Provisional Patent Application No. 63/040,295, filed on Jun. 17, 2020, which is hereby incorporated by reference in its entirety.

### STATEMENT REGARDING FEDERALLY SPONSORED RESEARCH OR DEVELOPMENT

Not Applicable

### BACKGROUND OF THE INVENTION

### Field of the Invention

The technology described herein relates to methods and devices for application of analyte-selective microneedle sensors to the skin of a wearer for physiological sensing of analytes.

### Description of the Related Art

The presentation of circulating biomarkers in a timely fashion remains a key aim in modern medical devices and chronic disease management, in particular. The most pertinent example of the need for low-latency biomarker or analyte quantification resides within the diabetes management domain and is addressed with continuous glucose monitoring systems (CGM or CGMS), which are widely used by individuals with insulin-dependent diabetes mellitus in order to inform dosing decisions involving the delivery of insulin or other pharmacologic agents.¹ Indeed, efficacy of CGM has been substantiated over the past decade in a multitude of clinical trials and end-user studies wherein noteworthy improvements in glycemic management (in contrast with self-monitoring of blood glucose via periodic fingerstick blood sampling) have been elucidated. However, surprisingly, uptake of these systems has been tepid and in direct contradiction to the strong outcomes-based evidence would suggest.² Tanenbaum and colleagues³ have explored the barriers to adherence and use of CGM in managing diabetes mellitus and have come to the conclusion that it is high system cost, meager reliability, and the overall poor user experience that limit widespread adoption of this transformational technology for diabetes management. Accordingly, enabling capabilities that aim at addressing these obstacles would position CGM for widespread adoption. One of these enabling capabilities that is ideally positioned to reducing cost, improving reliability, and augmenting the user experience resides in the devices and methods employed for application of CGM and analyte sensors, in general, to the skin of a wearer.

Current subcutaneously-implanted analyte-selective sensors are configured to execute the analyte sensing operation in the subcutaneous layer beneath the dermis, known as the subcutaneous adipose tissue. Likewise, intradermal analyte sensors, often embodied as microneedle arrays, execute the sensing operation more superficially, in the viable epidermis or dermis (papillary dermis or reticular dermis). In order to penetrate the skin and position the sensing element found within the analyte-selective sensor in the desired anatomical region (or strata), a mechanical applicator mechanism is often employed. These applicators typically contain stored potential energy in the form of a compressed mechanical element (i.e. spring, deformable material) or gas that is transferred to kinetic energy upon actuation by a user, causing the analyte-selective sensor retained within the said applicator to be applied to the user's skin at a defined force, velocity, displacement, momentum, and/or inertia. Penetrating to the desired strata of the skin is tantamount to proper analyte quantification, especially within the domain of microneedle-mediated analyte sensing. Indeed, the proper insertion of microneedle array-based analyte-selective sensors requires an extreme level of precision and, to date, has required the use of a spring- or piston-driven mechanical mechanism in order to store the requisite energy required for microneedle penetration of the skin in the form of potential energy. Indeed, the implementation of mechanical applicators is in direct contradiction to current efforts aimed at reducing system cost to increase accessibility of the technology, improving reliability, and reducing the level of complexity required to apply a sensor to enhance the user experience. A microneedle array analyte-selective sensor capable of application and subsequent insertion to the desired strata of the skin with only user-provided force would make substantial inroads to greater and more widespread adoption of CGM and body-worn analyte sensing, in general.

The prior art includes the following:

U.S. Pat. No. 9,789,249 for a Microneedle array applicator device and method of array application, which discloses an applicator device including a housing, an impactor for impacting a microneedle array and accelerating the microneedle array toward the target site, wherein the impactor is capable of moving along an arcuate path to move the microneedle array toward the target site.

U.S. Pat. No. 8,821,446 for Applicators for Microneedles which discloses a microneedle applicator is provided which has two roughly concentric portions which may be, for example, a solid disk and an annulus surrounding it.

U.S. Pat. No. 8,267,889 for a Low-profile microneedle array applicator, which discloses an applicator used to apply microneedle arrays to a mammal. In particular, an application device for applying a microneedle device to a skin surface comprising a flexible sheet having a raised central area attached to the microneedle device and a supporting member at or near the periphery of the flexible sheet, wherein the flexible sheet is configured such that it will undergo a stepwise motion in the direction orthogonal to the major plane of the sheet.

U.S. Pat. No. 9,687,640 for Applicators for microneedles, which discloses an applicator for a microprojection array is described. In one embodiment, the applicator comprises an energy-storing element.

U.S. patent Ser. No. 10/406,339 for a Force-controlled applicator for applying a microneedle device to skin, which discloses an applicator and method for applying a microneedle device to a skin surface.

U.S. patent Ser. No. 10/300,260 for an Applicator and method for applying a microneedle device to skin which discloses an applicator and method for applying a microneedle device to skin.

U.S. Pat. No. 8,579,862 for an Applicator for microneedle array which discloses a microneedle device which protects microneedle, has an easily portable shape, is free from such problems as breakage of small needles in the step of puncturing the skin with the microneedle, and ensures appropriate skin puncture for administering a drug.

U.S. patent Ser. No. 10/010,707 for an Integrated microneedle array delivery system, which discloses a low-profile system and methods for delivering a microneedle array.

U.S. Pat. No. 9,782,574 for a Force-controlled applicator for applying a microneedle device to skin, which discloses an applicator for applying a microneedle device to a skin surface. The applicator can include a microneedle device, a housing, and a connecting member.

U.S. Pat. No. 9,492,647 for a Microneedle array applicator and method for applying a microneedle array, which discloses a microneedle array applicator is configured to apply a microneedle array in cosmetic and medical applications.

U.S. Pat. No. 9,415,198 for a Microneedle patch applicator system, which discloses a method and apparatus for application of a microneedle patch to a skin surface of a patient includes use of an applicator.

U.S. Pat. No. 9,174,035 for a Microneedle array applicator and retainer, which discloses an applicator that has an elastic band to snap a microneedle array against skin with a predetermined force and velocity.

U.S. Pat. No. 9,119,945 for a Device for applying a microneedle array, which discloses a device for applying a microneedle array to a skin surface.

U.S. Pat. No. 8,758,298 for a Low-profile microneedle array applicator, which discloses an applicator used to apply microneedle arrays to a mammal.

Prior devices and methods to insert microneedle arrays into the dermal strata of a user largely leverage spring- or piston-driven applicator mechanisms to facilitate orthogonal acceleration of an embedded microneedle array towards the skin surface of a user with a specified force, velocity, and displacement profile. Additional previously described embodiments include applicators that comprise of retention of said sensor within a deformable membrane. The user applies a load to the top of the membrane until a set force is reached. Once a requisite force is attained, the deformable membrane collapses and the sensor is accelerated axial to the skin surface of a user. The deformable membrane can be constructed of a material that undergoes plastic deformation once a desired force is attained. In other embodiments, the deformable membrane is constructed from a metal and replicates the function of a dome spring. The geometry and material of said membrane can be modified to tune the desired deformation force and, in some cases, the geometry can be modified to augment said force, such that the force to actuate the membrane is less than the force applied by said membrane. In some embodiments, the membrane may be actuated directly by the user, or by means of a lever or a mating component, further augmenting the force generated by the membrane. In some embodiments, the application of orthogonal forces by the user, are translated into lateral forces by the geometry of the applicator, thus applying tension to the skin of a user in an effort to facilitate access to the desired skin strata.

### BRIEF SUMMARY OF THE INVENTION

The current invention teaches of methods and devices enabling the insertion of a microneedle array-based analyte-selective sensors to the desired strata of the viable epidermis or dermis with user-supplied force. The aim of this solution is to provide a method for a user to insert an analyte-selective microneedle array sensor into a desired strata of a user's skin while ensuring that the application force, velocity and insertion angle at impact are controlled. In some embodiments, application is achieved solely with user-supplied force, while in other embodiments, user-supplied force is augmented with force from an energy storage device (i.e. spring). User-supplied force is controlled by a mechanism wherein the sensor or sensor carrier is retained by a gating or detent feature that requires a modest force to overcome. The impact velocity can be controlled by the force required to overcome the gating or detent feature and the travel distance. The angle of insertion is controlled by guide elements found within the application mechanism and/or analyte sensor device. In other embodiments, this solution is enabled by a mechanism wherein an armature retaining a sensor by an interference fit is deployed by a modest user-supplied force and thereby accelerated to a defined impact force and velocity specification. The proximal extremity of said armature is meant to pivot about a hinge, joint, or shaft and may be, optionally, aided by a torsion element such as a spring or elastic member. In some embodiments, the application mechanism is configured to render the skin at the application site immobile or apply tension to the skin at the application site to reduce elasticity and improve reliability of insertion. Advantages of these approaches compared with prior art devices and methods for microneedle application include simplified application process and thereby user experience, lower cost of goods due to reduced bill of materials, reduced package size hence logistics and shelf-space, less waste, and improved reliability due to the reduced count of mechanical components.

One aspect of the present invention is an applicator device configured for the insertion of an analyte-selective microneedle array sensor into a dermal stratum of a user. The device comprises a body portion configured to be grasped with a hand of said user, a carrier configured to retain said sensor and accelerate sensor during deployment towards the skin surface of said user, a shaft at a proximal end of said carrier configured to enable carrier to undergo radial motion about said shaft, a spring plunger configured to apply an engineering fit to retain carrier in a first position, and a release mechanism configured to deform its shape upon compression by said spring plunger. A user-directed application of a specified force to the carrier causes the spring plunger to retract and the release mechanism to return to its native shape, thereby to effect the acceleration of the microneedle array sensor device in an arc-like motion about said shaft and towards the skin surface of a user with a specified impact force and velocity. The insertion depth beneath the skin surface of a user is dependent on the velocity and mass (momentum) of the microneedle array when it impacts the skin.

Another aspect of the present invention is a method for the insertion of an analyte-selective microneedle array sensor into a dermal stratum of a user. The method includes positioning an applicator mechanism containing said analyte-selective sensor on the skin of a user. The method also includes applying a minimum force on a carrier within said applicator mechanism, thereby causing a spring plunger to retract and a deformed release mechanism to return to its native shape. The decompression of said release mechanism effects the acceleration of the microneedle array sensor device in an arc-like motion about a shaft from a first position within said applicator mechanism and towards the skin surface of said user with a specified impact force and velocity. The insertion depth beneath the skin surface of a user is dependent on the velocity and mass (momentum) of the microneedle array when it impacts the skin.

Yet another aspect of the present invention is a sterile barrier package applicator device. The sterile barrier package applicator device comprises a first aperture, a second aperture, a body portion, an analyte-selective microneedle array sensor retained by an engineering fit in a first position within said body portion, the non-sensing surface of said analyte-selective microneedle array positioned in proximity to said first aperture, and a film disposed over said second aperture of said sterile barrier package, said film configured to be removed by a user. A user-directed application of a minimum force to the non-sensing surface of said analyte-selective microneedle array compromises said engineering fit, thereby to effect the acceleration of the microneedle array sensor device in a linear motion from a first position to a second position and towards the skin surface of a user with a specified impact force, velocity, and angle of insertion. The insertion depth beneath the skin surface of a user is dependent on the velocity and mass (momentum) of the microneedle array when it impacts the skin.

Yet another aspect of the present invention is a method for the insertion of an analyte-selective microneedle array sensor into a dermal stratum of a user by means of a sterile barrier package applicator containing a first aperture, second aperture, and body portion. The method includes removing a film disposed over said second aperture of said sterile barrier package applicator. The method also includes positioning second aperture of said sterile barrier package applicator containing said analyte-selective sensor on the skin of a user. The method also includes applying a minimum force to the non-sensing surface of said analyte-selective microneedle array sensor. The application of a minimum force by a user compromises an engineering fit retaining said analyte-selective microneedle array sensor to said body portion, thereby to effect the acceleration of the microneedle array sensor device in a linear motion from a first position to a second position and towards the skin surface of a user with a specified impact force, velocity, and angle of insertion.

Yet another aspect of the present invention is an applicator device configured for the insertion of an analyte-selective microneedle array sensor into a dermal stratum of a user. The device comprises a body portion configured to be grasped with a hand of said user, a recessed actuation portion configured to be pressed with a finger of said user, a carrier configured to retain said sensor and accelerate sensor during deployment towards the skin surface of said user, a gating feature configured to prevent carrier movement until a minimum force is applied, and a disengagement feature configured to release the sensor upon deployment. A user-directed application of a specified force to the actuation area causes the carrier to overcome the gating feature, thereby to effect the acceleration of the microneedle array sensor device towards the skin surface of a user with a specified impact force and velocity.

The microneedle array sensor is preferably an electrochemical, electrooptical, or fully electronic device. The microneedle array sensor is preferably configured to measure at least one of an endogenous or exogenous biochemical agent, metabolite, drug, pharmacologic, biological, or medicament indicative of a particular physiological or metabolic state in a physiological fluid of a user. The microneedle array sensor preferably contains a plurality of microneedles, each possessing a vertical extent between 200 and 2000 μm. The microneedle array sensor preferably contains a housing containing a power source, electronic measurement circuitry, a microprocessor, and a wireless transmitter. The microneedle array sensor is preferably configured with a skin-facing adhesive intended to adhere the said sensor to the skin surface of the wearer for an intended wear duration.

The dermal stratum is the viable epidermis, papillary dermis, or reticular dermis. The body portion preferably features at least one flange to enhance retention by hand of the user.

The carrier is configured to retain the microneedle array sensor by means of at least one of an interference fit, friction fit, press fit, clearance fit, and a location fit. The shaft is preferably a hinge. Torsion is preferably applied to the shaft, and is preferably achieved by a flexible elastic member. The flexible elastic member is preferably a torsion spring, leaf spring, sprung metal member, or sprung plastic member. The spring plunger is preferably a ball nose spring. An engineering fit is at least one of an interference fit, friction fit, press fit, clearance fit, and a location fit. The first position is preferably recessed within the body portion. The release mechanism is a rigid or elastic member. The release mechanism is preferably configured to further apply retention to the sensor. The user-directed application of a specified force is preferably between 0.3 N and 30 N. The impact force is preferably between 0.3 N and 30 N. The velocity is preferably between 0.15 m/s and 15 m/s.

Having briefly described the present invention, the above and further objects, features and advantages thereof will be recognized by those skilled in the pertinent art from the following detailed description of the invention when taken in conjunction with the accompanying drawings.

### BRIEF DESCRIPTION OF THE SEVERAL VIEWS OF THE DRAWINGS

FIG. 1 is an illustration of a prior art needle-/cannula-based analyte-selective sensor (left) configured for the quantification of glucose in the subcutis and a microneedle array-based analyte-selective sensor (right) configured for the quantification of glucose in the dermis.

FIG. 2 is a magnified representation of FIG. 1.

FIG. 3 is an illustration of a microneedle array-based analyte-selective sensor proposed in the current invention; the sensing element (electrode) is located in the distal region of the analyte-selective sensor and is intended to execute the measurement operation in the viable epidermis or dermis.

FIG. 4 is a pictorial representation of a conventional wire-/needle-/cannula-based analyte-selective sensor configured to operate within the subcutaneous tissue (left) and an analyte-selective sensor configured to operate within the dermis (right). It should be noted that the sensing element contained within the analyte-selective sensor (right) is located in the papillary dermis.

FIG. 5 is a pictorial representation of a cross-section of the skin delineating the anatomical location of the papillary plexus and structures contained therein.

FIG. 6 is a pictorial representation of a cross-section of the skin delineating the anatomical location of the superficial and dermal plexus and structures contained therein. Source: Rose L. Hamm: Text and Atlas of Wound Diagnosis and Treatment. © McGraw-Hill Education.

FIG. 7A is an illustration of a current subcutaneously-implanted continuous glucose monitor (CGM) system with its corresponding mechanical applicator shown. Dexcom® G6™ (applicator, left)

FIG. 7B is an illustration of a current subcutaneously-implanted continuous glucose monitor (CGM) system with its corresponding mechanical applicator shown. Abbott® Freestyle Libre™ bottom (applicator, left).

FIG. 8 is an illustration of a microneedle array analyte-selective sensor undergoing application by means of a user-directed force.

FIG. 9 is an illustration of an exemplary applicator device enabling the application of microneedle array analyte-selective sensors to the skin of a user using a radial acceleration about a shaft.

FIG. 9A is a magnified representation of FIG. 9.

FIG. 10 is an illustration of a microneedle array analyte-selective sensor loaded into the applicator device of FIG. 9.

FIG. 11 is a photograph of priming the applicator device of FIG. 9, loaded with a microneedle array analyte-selective sensor, to enable application of said sensor with a desired force and velocity.

FIG. 11A is an illustration of the applicator device of FIG. 9, the auto release component deforming.

FIG. 12 is an illustration of applying the microneedle array analyte-selective sensor using the applicator of FIG. 9.

FIG. 13 is an exploded view of an exemplary applicator device of FIG. 9, delineating the major components.

FIG. 13A is an illustration of an exemplary applicator device of FIG. 13 assembled.

FIG. 14 is an illustration of high-level mechanical representation of the exemplary application mechanism wherein the radial acceleration of the microneedle array is imparted by the application of a user-directed force, Fₚᵣₑₛₛ, which is greater than the product of the spring constant k₂ and displacement Δx of the ball spring.

FIG. 15 is a block diagram of a peel-and-stick method intended to apply a microneedle array-based analyte-selective sensor to the skin of a wearer.

FIG. 16A is a top plan view of an embodiment of the device of the invention.

FIG. 16B is a bottom plan view of the device of FIG. 16A.

FIG. 16C is a side view of the device of FIG. 16A.

FIG. 16D is a bottom perspective view of the device of FIG. 16A.

FIG. 16E is a top perspective view of the device of FIG. 16A.

FIG. 16F is a top perspective view of the device of FIG. 16A.

FIG. 16G is a bottom perspective view of the device of FIG. 16A with the adhesive liner removed.

FIG. 16H is an exploded view of the device of FIG. 16A.

FIG. 17A is an illustration of a flex-compression method intended to apply a microneedle array-based analyte-selective sensor to the skin of a wearer at a specified force, velocity, and displacement. Sensor in its unperturbed state.

FIG. 17B is an illustration of a flex-compression method of FIG. 17A with the sensor undergoing the application of a specified force provided by a user.

FIG. 18A is a top plan view of an embodiment of the device of the invention.

FIG. 18B is a side view of the device of FIG. 18A.

FIG. 18C is a bottom plan view of the device of FIG. 18A.

FIG. 18D is a top perspective view of the device of FIG. 18A.

FIG. 18E is an interior perspective view of the device of FIG. 18A.

FIG. 18F is a bottom perspective view of the device of FIG. 18A.

FIG. 18G is a perspective view of the device of FIG. 18A.

FIG. 19A is an illustration of a flex-compression method with a mechanical detent feature intended to apply a microneedle array-based analyte-selective sensor to the skin of a wearer at a specified force, velocity, and displacement. Sensor in its unperturbed state.

FIG. 19B is an illustration of a flex-compression method of FIG. 19A with the sensor undergoing the application of a specified force provided by a user.

FIG. 20 is a sectional view of the flex-compression method with a mechanical detent feature intended to apply a microneedle array-based analyte-selective sensor to the skin of a wearer.

FIG. 21A is a sectional view of an embodiment of the device of the invention with the sensor in its unperturbed state.

FIG. 21B the device of FIG. 21A with the sensor undergoing the application of a specified force provided by a user.

FIG. 22 is a cutaway view of the alternative compression method of application of a microneedle array-based analyte-selective sensor to the skin of a wearer at a specified force, velocity, and displacement.

FIG. 23A is a top plan view of an embodiment of the device of the invention.

FIG. 23B is a side view of the device of FIG. 23A.

FIG. 23C is a bottom plan view of the device of FIG. 23A.

FIG. 24A is a perspective view of an embodiment of the device of the invention.

FIG. 24B is a perspective view of the device of FIG. 24A with the cover removed.

FIG. 25A is an illustration of an embodiment of the device of the invention with the sensor in its unperturbed state.

FIG. 25B is an illustration of the device of FIG. 25A with the sensor undergoing the application of a specified force provided by a user.

FIG. 25C is an illustration of the device of FIG. 25A with the sensor retained on the skin of a user following the ‘press-on’/pressure application process applied by said user.

FIG. 26A is a top plan view of an embodiment of the device of the invention.

FIG. 26B is a bottom plan view of the device of FIG. 26A.

FIG. 26C is a perspective view of the device of FIG. 26A.

FIG. 26D is a side view of the device of FIG. 26A.

FIG. 26E is an exploded view of the device of FIG. 26A.

FIG. 27A is a bottom perspective view of the device of FIG. 26A.

FIG. 27B is a perspective view of the device of FIG. 27A with the cover being removed.

FIG. 28A is a top plan view of an embodiment of the device of the invention.

FIG. 28B is a perspective view of the device of FIG. 28A.

FIG. 28C is a side view of the device of FIG. 28A.

FIG. 28D is a side view of the device of FIG. 28A with the sensor deployed

FIG. 29A is a top plan view of an embodiment of the device of the invention.

FIG. 29B is a perspective view of the device of FIG. 28A.

FIG. 29C is a side view of the device of FIG. 29A.

FIG. 29D is a side view of the device of FIG. 29A with the sensor deployed

FIG. 30A is a top plan view of an embodiment of the device of the invention.

FIG. 30B is a front perspective view of the device of FIG. 30A.

FIG. 30C is a back perspective view of the device of FIG. 30A.

FIG. 30D is the device of FIG. 30C in a deployed position.

FIG. 30E is a front plan view of the device of FIG. 30A.

FIG. 30F is a sectional view of the device of 30E.

FIG. 30G is a sectional view of the device of 30E.

FIG. 31A is a top plan view of an embodiment of the device of the invention.

FIG. 31B is a perspective view of the device of FIG. 31A.

FIG. 31C is a side view of the device of FIG. 31A in a cocked position.

FIG. 31D is a sectional view of the device of FIG. 31C.

FIG. 31E is a side view of the device of FIG. 31A in a deployed position.

FIG. 31F is a sectional view of the device of FIG. 31E.

FIG. 32A is a top plan view of an embodiment of the device of the invention.

FIG. 32B is a side view of the device of FIG. 32A.

FIG. 32C is a top perspective view of the device of FIG. 32A.

FIG. 32D is the piston of the device of FIG. 32A.

FIG. 32E is a perspective view of the device of FIG. 32A.

FIG. 32F is a bottom view of the device of 32A.

FIG. 32G is a bottom perspective view of the device of 32A after deploying.

FIG. 32H is a bottom perspective view of the device of 32A in a loaded position.

FIG. 33A is a top plan view of an embodiment of the device of the invention.

FIG. 33B is a perspective view of the device of FIG. 33A.

FIG. 33C is a side view of the device of FIG. 33A in a cocked position.

FIG. 33D is a sectional view of the device of FIG. 33C.

FIG. 33E is a side view of the device of FIG. 33A in a deployed position.

FIG. 33F is a sectional view of the device of FIG. 33E.

FIG. 34 is a time-series dataset substantiating the ability of a microneedle array-based glucose-selective sensor applied without a mechanical application mechanism to track glucose in the dermis of a wearer in a quantitative fashion. Correlate glucose measures (Dexcom® G5™ CGM, YSI® Model 2300 Electrochemical Analyzer™) as well as periodic calibrations with fingerstick blood samples (Bayer® Contour NEXT™) is provided in the plot.

FIG. 35 is a representative image from an optical coherence tomography (OCT) analysis of an array with seven microneedles with strain applied to the skin during application.

FIG. 36 is a representative image from an optical coherence tomography (OCT) analysis of an array with seven microneedles with the skin in a native state (no external strain imposed).

FIG. 37 is a representative image from an optical coherence tomography (OCT) analysis of an array with thirty-seven microneedles with the skin in a native state (no external strain imposed).

FIG. 38 is a representative image from an optical coherence tomography (OCT) analysis of an array with thirty-seven microneedles with the skin in a native state (no external strain imposed).

FIG. 39 is a representative image from an optical coherence tomography (OCT) analysis of an array with thirty-seven microneedles with strain applied to the skin during application.

FIG. 40 is a representative image from an optical coherence tomography (OCT) analysis of an array with thirty-seven microneedles applied with a mechanical applicator with strain applied to the skin during application.

FIG. 41A is a table of experimental configurations of microneedle array analyte-selective sensors applied to a wearer without the aid of a mechanical applicator mechanism.

FIG. 41B is a table of a Control configuration of a microneedle array analyte-selective sensors applied to a wearer with the aid of a mechanical applicator mechanism.

FIG. 42 is a representative bar chart and descriptive statistics of the insertion depth below the skin surface embodied by each microneedle array analyte-selective sensor configuration studied.

FIG. 43 is a representative bar chart and descriptive statistics of the insertion depth below the epidermal-dermal junction embodied by each microneedle array analyte-selective sensor configuration studied.

FIG. 44 is a histogram illustrating the distribution of insertion depth embodied by each microneedle array analyte-selective sensor configuration studied.

FIG. 45 is a block diagram of a method of the invention.

FIG. 46 is a block diagram of a method of the invention.

FIG. 47 is a block diagram of a method of the invention.

### DETAILED DESCRIPTION OF THE INVENTION

Current subcutaneously-implanted analyte-selective sensors have enjoyed much use in continuous physiological monitoring, driven primarily by the challenge of glucose quantification for diabetes applications. Configured to engage in the measurement of physiological analytes in the subcutaneous layer beneath the dermis, these analyte-selective sensors are inserted to this anatomic region by means of spring- or piston-driven applicators, which ensheathes the sensing contingent with a retractable cannula. New developments in the field of dermal sensing, and microneedle-mediated analyte-selective sensing, in particular, facilitate simplified methods of application such that said cannula is no longer required to insert a sensor to the desired anatomical region.

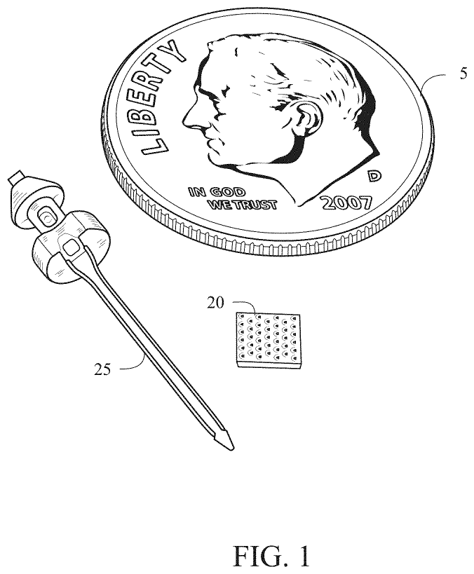

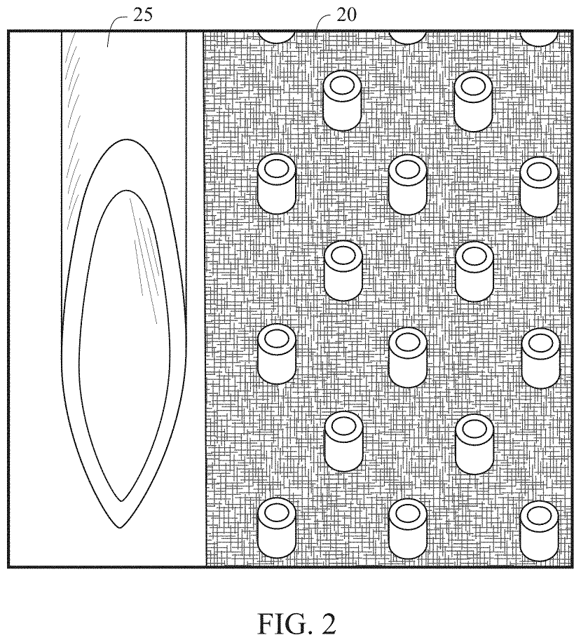

FIG. 1 shows a prior art needle-/cannula-based analyte-selective sensor 25 configured for the quantification of glucose in the subcutis and a microneedle array-based analyte-selective sensor 20 configured for the quantification of glucose in the dermis. A dime 5 is shown to demonstrate the size of the sensors. FIG. 2 is a magnified representation of FIG. 1

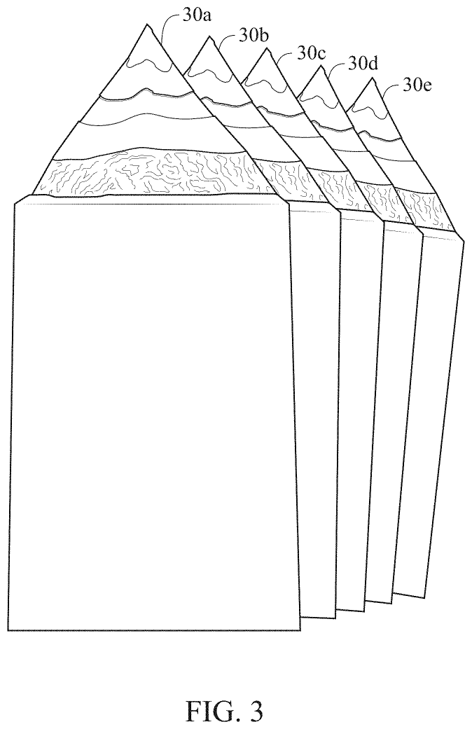

FIG. 3 shows a microneedle array-based analyte-selective sensor proposed in the current invention; the sensing elements 30a-e are located in the distal region of the analyte-selective sensor and is intended to execute the measurement operation in the viable epidermis or dermis.

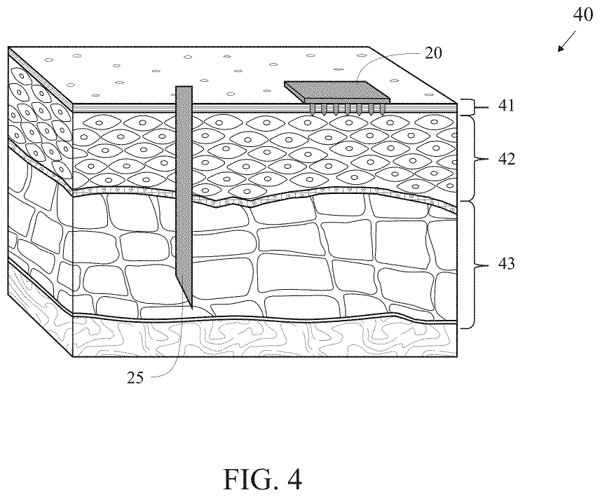

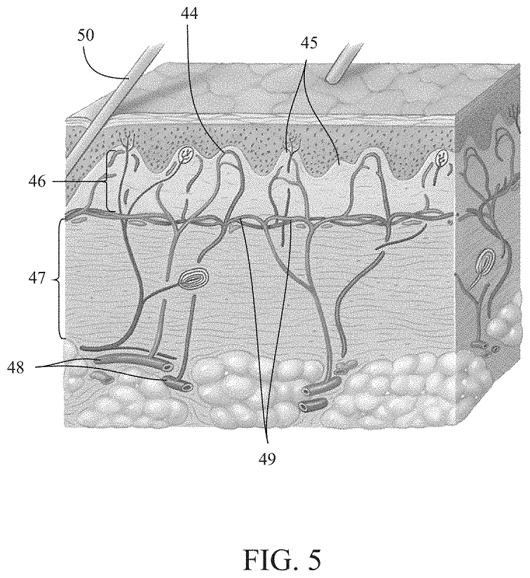

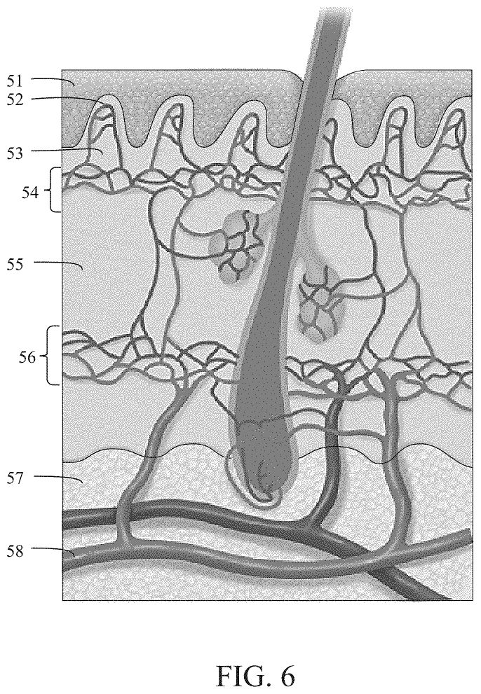

However, owing to the unique dynamics of insertion of microneedles into the skin, the design of these microneedle applicators requires that great care be taken to design the application mechanism to overcome the viscoelastic response of the skin. Inline with this aim, a cohesive set of design requirements must be pursued to achieve a minimum specified impact force and velocity to overcome said viscoelastic response. Furthermore, displacement and angle of incidence are also of fundamental performance in order to ensure access to the desired skin strata of the viable epidermis or dermis. FIGS. 4-6 are pictorial representations of cross-sections of the skin. FIG. 4 shows skin structure 40 and a conventional wire-/needle-/cannula-based 25 analyte-selective sensor configured to operate within the subcutaneous tissue 43 and an analyte-selective sensor 20 configured to operate within the dermis 42, beneath the epidermis 41. It should be noted that the sensing element contained within the analyte-selective sensor 20 is located in the papillary dermis. FIG. 5 shows the skin delineating the anatomical location of the papillary plexus 44 and structures contained therein. The following are shown: hair 50, capillary loop of papillary plexus 44, dermal papillae 45, papillary layer 46, reticular layer 47, cutaneous plexus 48, and subpapillary plexus 49. FIG. 6 shows the skin delineating the anatomical location of the superficial and dermal plexus and structures contained therein, including the following: epidermis 51, capillary loop system 52, papillary dermis 53, superficial vascular plexus 54, reticular dermis 55, deep vascular plexus 56, subcutaneous fat 57, and subcutaneous arteries 58.

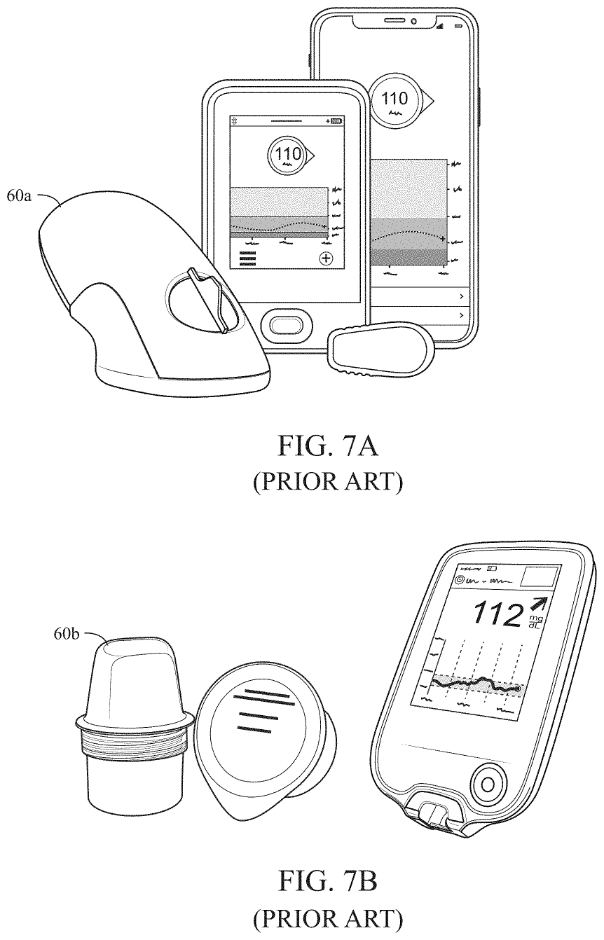

Indeed, analyte-selective microneedle array sensors comprise of sharp, protruded sensing elements and can be easily deployed just below the surface of the skin, enabling insertion by a user-supplied force (without necessarily requiring an applicator mechanism). However, in order to reliably insert microneedle array-based analyte-selective sensors into the desired skin strata, it is necessary to control one or more critical application parameters including force, velocity, angle of insertion, and skin tension. Due to expected variation among a user population, it is necessary to control one or more of these critical parameters during the application process to ensure reliable application of said sensor and concomitant insertion of sensing elements into the desired skin strata. The noteworthy benefit of these solutions over the prior art include the reduced number of mechanical constituents, which is commensurate with the requirements of a high volume, low cost product. The simplified design reduces the size and complexity of the application mechanism, which is directly related to cost. The current invention also provides for an improved user experience; the user can simply ‘press’ the sensor on the skin surface rather than use a cumbersome applicator for said application. Many of the applicators described in the prior art, such as those shown in FIGS. 7A-7B, comprise multiple components, some of which are utilized to store potential energy, which results in increased bill of materials and cost of goods. This design may reduce the system down to a minimum set of extricable components, many of which may be injection molded, resulting in very low cost production.

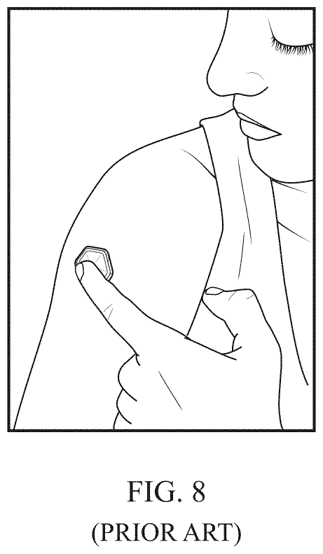

FIGS. 7A-7B are an illustration of two current subcutaneously-implanted continuous glucose monitor (CGM) systems with their corresponding mechanical applicators 60a-b shown. The Dexcom® G6™ system is shown in FIG. 7A, and the Abbott® Freestyle Libre™ system is shown in FIG. 7B. Another prior art method is shown in FIG. 8, the application of the sensor by means of a user-directed force.

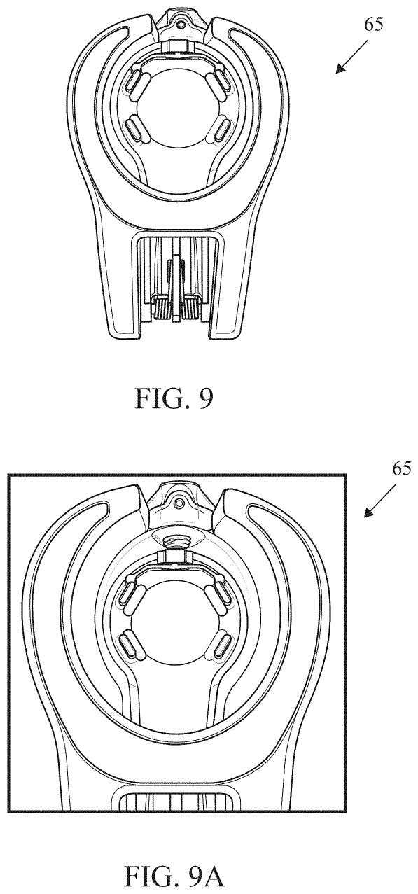

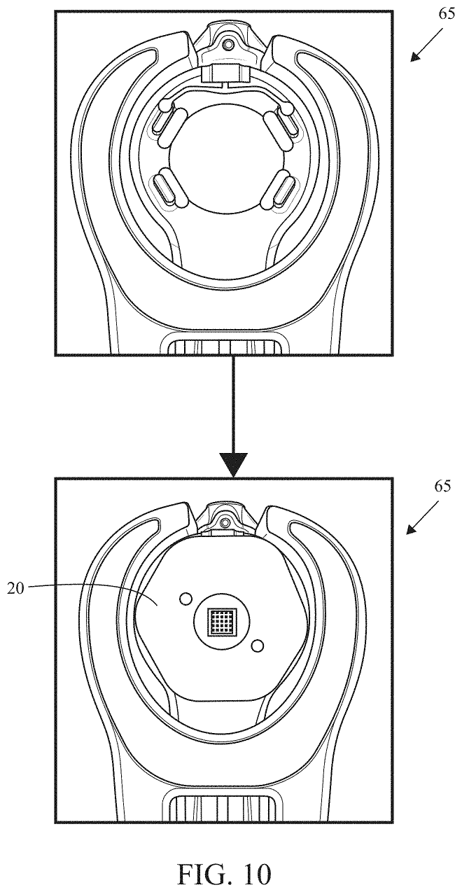

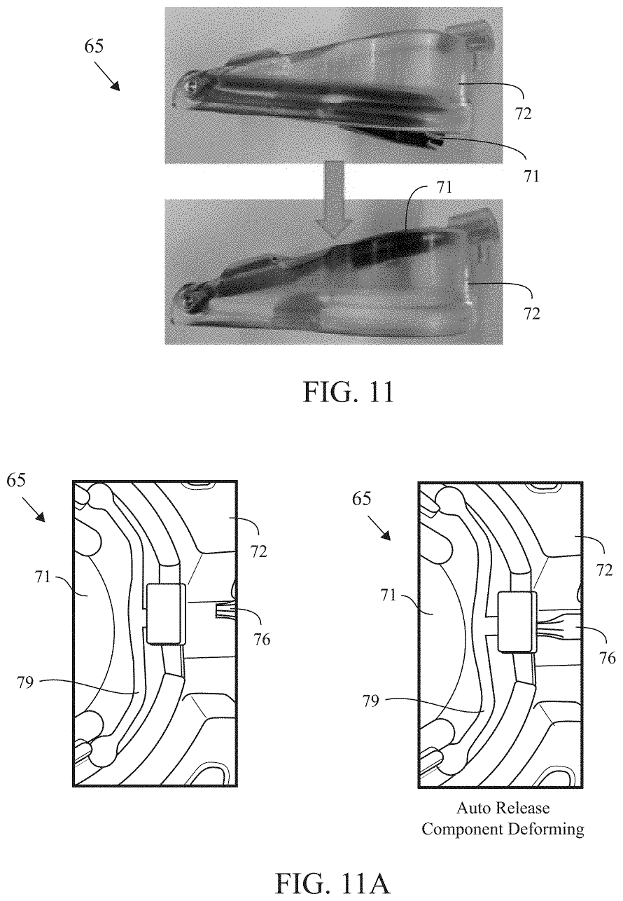

The applicator device 65 and applicator mechanism taught in this disclosure to effect the application of an analyte-selective microneedle array sensor to the skin surface of a user concern the implementation of an armature 71 (otherwise referred to as a ‘carrier’) to which said sensor is retained with an engineering fit. Said armature 71 is configured to undergo a radial or arc-like acceleration upon an actuation or deployment event by a user. FIGS. 9-13A show an exemplary applicator device 65. FIG. 10 shows the sensor 20 loaded into the applicator device 65. FIG. 11 is a photograph of priming the applicator device 65, loaded with a microneedle array analyte-selective sensor within the carrier 71. The top figure shows the carrier 71 in a deployed position and the bottom figure shows the carrier 71 in a relaxed position after releasing the sensor. In FIG. 11A, the auto release component deforming is shown.

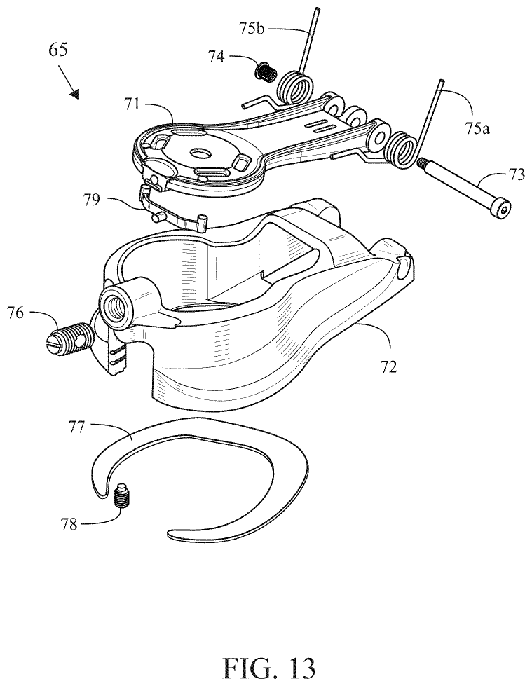

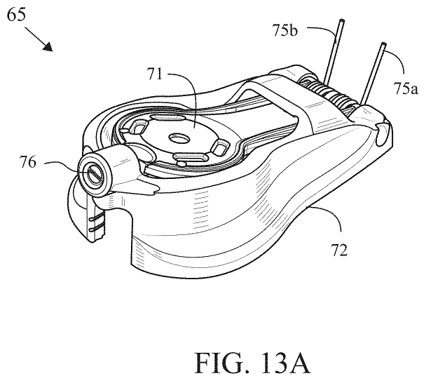

FIG. 13 shows the device 65 delineating the major components in an exploded view. The applicator device 65 is composed of a holder 72, carrier 71, shaft 73, threaded insert 74, torsion springs 75a-b, ball nose/spring plunger 76, rubber pad 77, nylon tip/set screw 78, and an auto release 79. FIG. 13A shows the device 65 assembled.

This is enabled by means of a shaft 73 or pivot, which provides for the pivot point about which said radial or arc-like motion is effectuated. Said shaft 73 or pivot is optionally torsioned by a spring 75 or elastic member, which serves to store kinetic energy in the form of potential energy. In alternative embodiments, the armature/carrier is maintained at a prescribed distance from the skin surface by a gating feature that can be overcome with a defined force. When the user applies a force that is below the minimum required to reliably insert the sensor, the sensor remains retained within the carrier by the gating feature. Upon application of a minimum force by a user on said armature (actuation or deployment event), said potential energy transfers to kinetic energy as an embedded microneedle array affixed to said armature is accelerated in either an axial or radial/arc-like trajectory from a first position wholly within said applicator mechanism to a second position in which the said analyte-selective microneedle array sensor is applied to the skin to effect the insertion of the microneedle constituents of the said sensor into a user's dermal stratum at a specified impact force, velocity, and angle. In some embodiments, the user's skin is either maintained in a fixed position or tensioned to control that aspect. The dermal stratum can either comprise the viable epidermis or dermis and in the vicinity of the papillary plexus, subpapillary plexus, or dermal plexus.

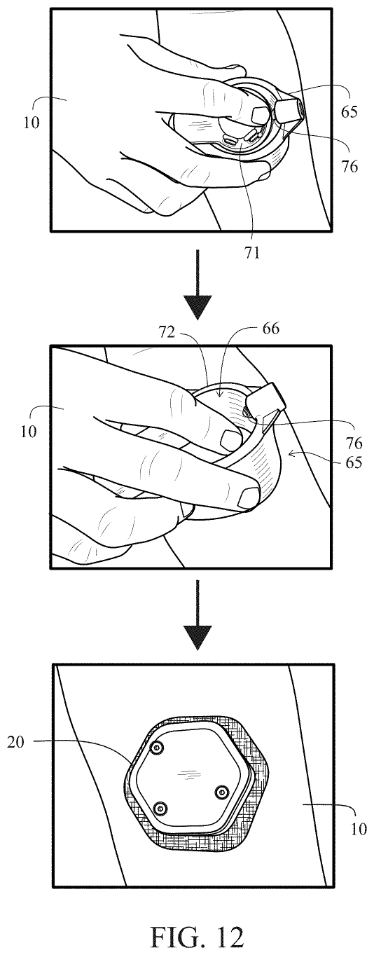

Applying the sensor 20 using the applicator 65 is shown in FIG. 12. The applicator device 65 is composed of a body portion, a holder 72, configured to be grasped with a hand of the user 10. The user 10 positions the device 65 on the skin. The holder 72 has a recessed actuation portion 66 configured to be pressed with a finger of the user 10. The holder 72 houses a carrier 71 configured to retain the sensor and accelerate sensor during deployment towards the skin surface of the user 10 as it is pressed. A gating feature using the ball nose/spring plunger 76 is configured to prevent carrier movement until a minimum force is applied. After pressing, a disengagement feature is configured to release the sensor upon deployment using the auto release 79, as shown in FIG. 13, leaving the sensor 20 at the desired position on the user 10.

In radial application embodiments, the acceleration a of the sensor to the skin of the user is given by the time-derivative of the velocity v, namely:

$$a = \frac{d v}{d t} = \frac{cos ( θ )}{{m}_{a}} ( {F}_{u s e r} + {k}_{a} h ) + g$$

wherein t refers to time, θ is the angle between the armature and the skin of a user, mₐ is the mass of the armature, Fᵤₛₑᵣ is the force applied by the user, kₐ is the constant of the torsion applied to the shaft at the proximal extremity of the armature, h is the height of the armature above the skin surface, and g is the acceleration due to gravity. This equation may be integrated to yield the time-dependent velocity of the sensor:

$$v ( t ) = {\int }_{0}^{t} \{ \frac{cos ( θ )}{{m}_{a}} ( {F}_{u s e r} + {k}_{a} h ) + g \} d t$$

Provided that the sensor undergoes radial motion, the instantaneous acceleration may be determined by the formula:

$$\vec{a} = \frac{{\vec{v}}^{2}}{r}$$

where r is the length of the armature.

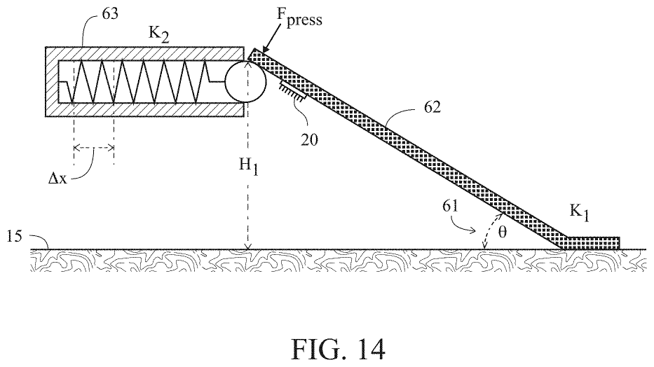

FIG. 14 shows a high-level mechanical representation of the mechanism wherein the radial acceleration of the microneedle array 20 is imparted by the application of a user-directed force, Fₚᵣₑₛₛ, which is greater than the product of the spring constant K₂ and displacement Δx of the ball spring 63. θ is the angle 61 between the armature 62 and the skin 15 of a user, kₐ is the constant K₁ of the torsion applied to the shaft at the proximal extremity of the armature 62, and h is the height H₁ of the armature 62 above the skin surface 15.

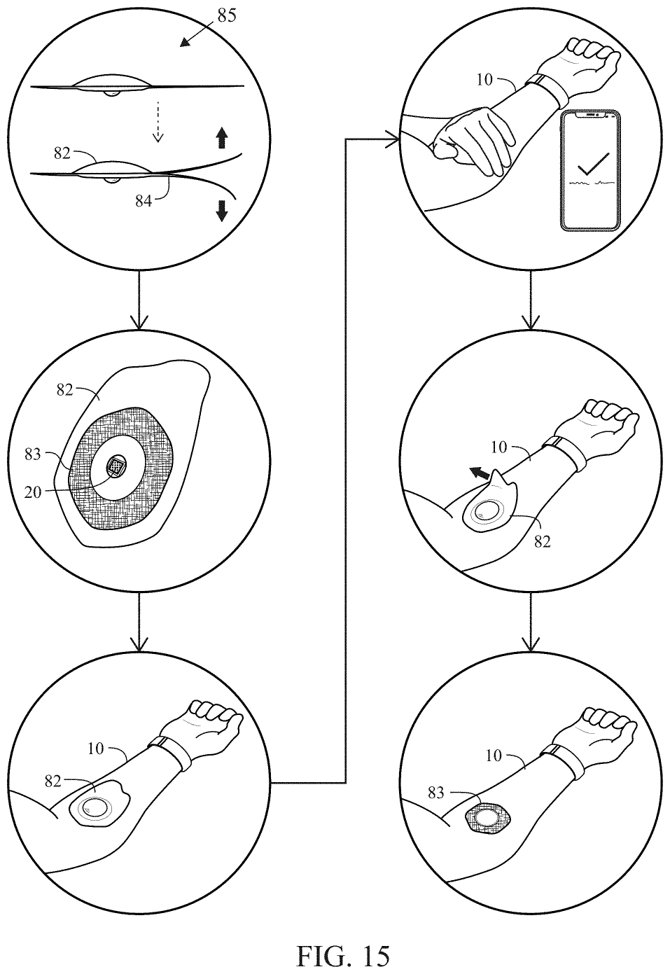

FIG. 15 is a block diagram of a peel-and-stick method intended to apply a sensor 20 to the skin of a wearer. The peel-and-stick device 85 is comprised of sterile paper 82, biotape 83 which contains the sensor 20, nonstick paper 84, and a protective bioplastic dome 81 which protects the sensor 20. The protective paper liner 84 is removed, exposing the application side (anterior surface) of the biotape 83 and sensor 20 which are still attached to the sterile paper 82. The user/wearer 10 applies the sterile paper 82 to the skin surface, with the application side (biotape 83) towards the skin. The user 10 presses gently on the sterile paper 82 and then removes the sterile paper liner 82 to expose the posterior surface of the sensor 20.

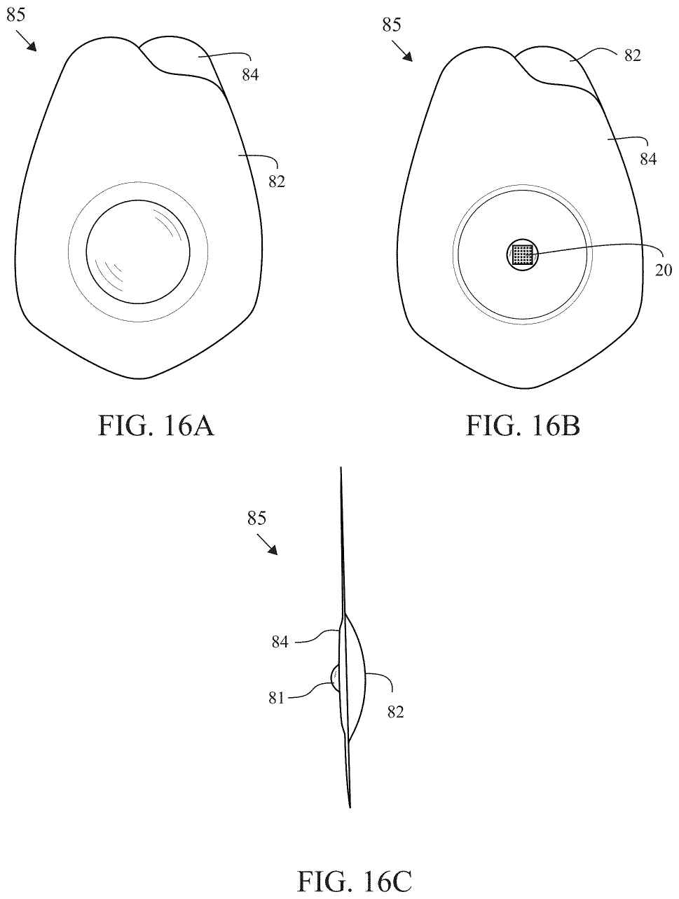

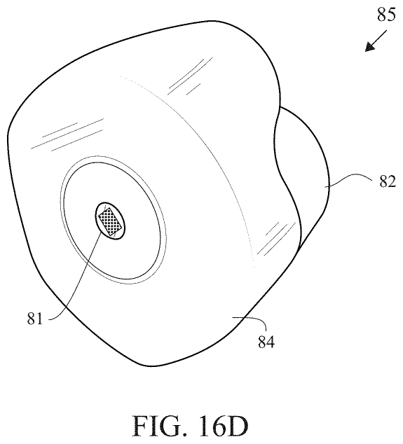

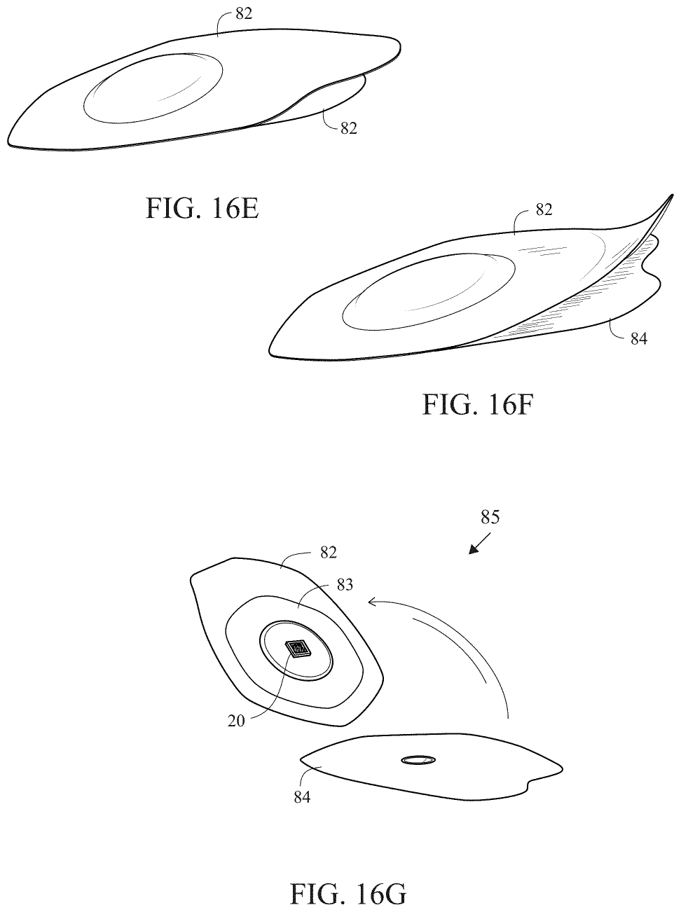

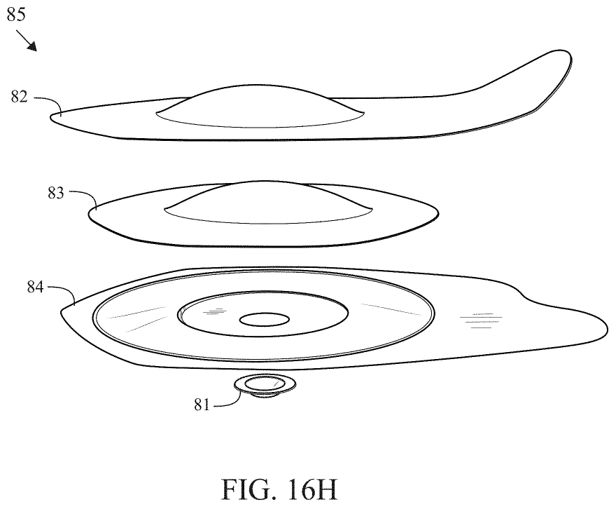

FIGS. 16A-H show perspective and exploded views of the peel-and-stick device 85. FIG. 16F shows the device 85 as it is peeled, and the two parts of the device 85 separated and peeled apart is shown in FIG. 16G.

Embedded tensioner embodiments:

It is generally accepted in the microneedle development and application industry that skin tensioning/stretching improves the efficacy of microneedle insertion. Other micro-needle application devices typically stretch the skin outside of the perimeter of the microneedle and its carrying housing.

This stretching is typically executed as a preliminary step, before insertion, and the skin is held stretched during the process of insertion. The skin stretching mechanism is typically an independently actuated motion. The displacement of the stretcher, amount of stretch in the skin, can be graphed as a typical stress-strain curve and is a percentage (approximately 30% if stretching on one axis, which is typical for an effective stretch) and therefore the mechanism required to perform the stretching motion around the perimeter of the microneedle housing is relatively large and requires multiple actuating parts. One problem with stretching the skin over this relatively large macro-area outside of the sensor is that it can cause pain, and second problem is that some areas of the body such as the lower arm of a smaller person has such a radius of curvature that a macro-stretcher can be rendered ineffective.

The primary objectives of Embedded tensioner embodiments are: to minimize the total mass and area of the skin to be stretched; to stretch the skin for a very short period of time rather than hold the skin in a stretched (painful) position during insertion; and to auto-stretch the skin with no secondary action required by the applicator's operator.

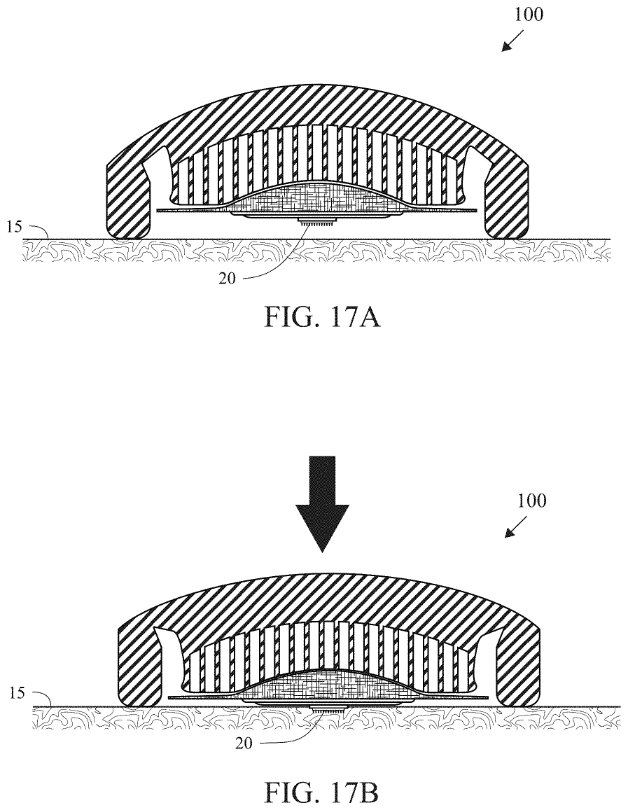

FIG. 17A shows a device 100 of a flex-compression method with the sensor 20 in its unperturbed state. FIG. 17B shows the sensor 20 undergoing the application of a specified force provided by a user, applying the sensor 20 to the skin 15 of the user.

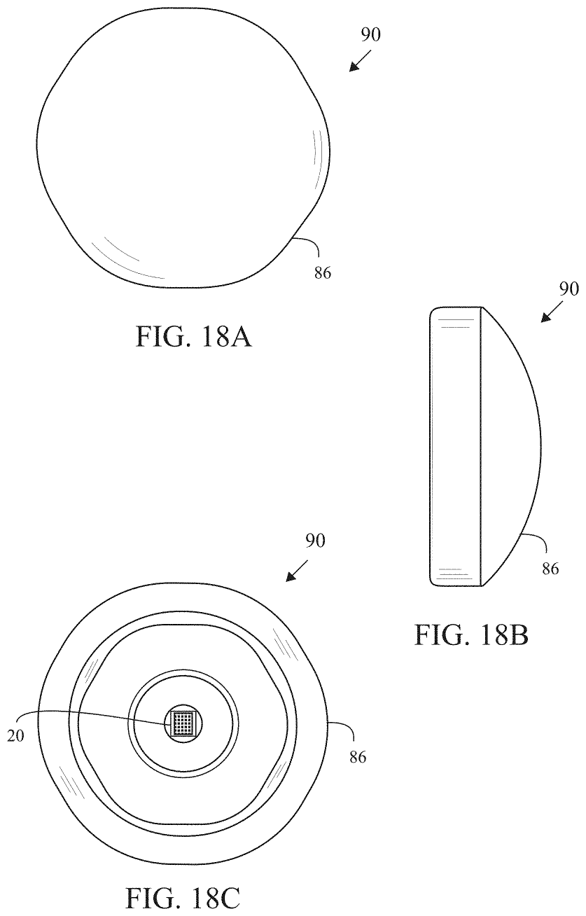

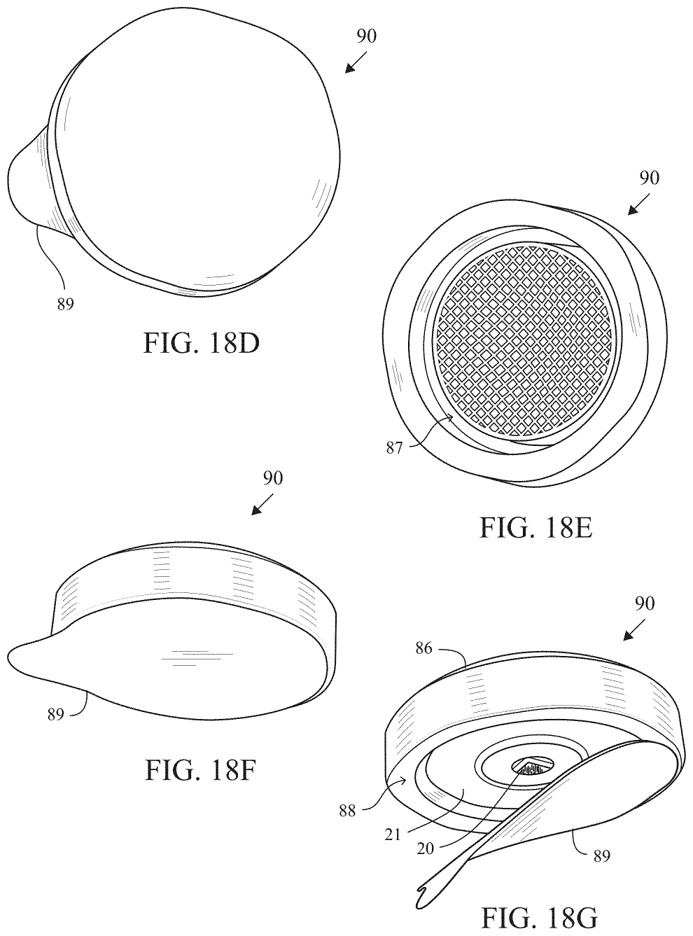

FIGS. 18A-G are perspective views of a sterile barrier package applicator device 90.

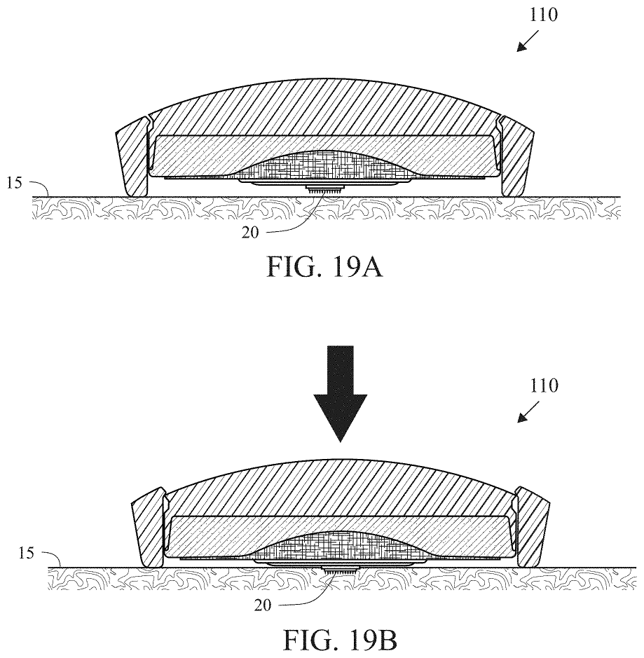

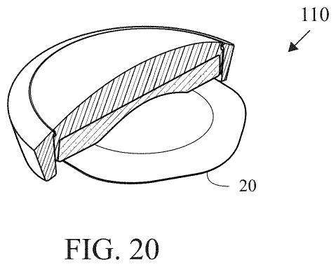

FIGS. 19A-20 show a device 110 of a flex-compression method. In FIG. 19A, the sensor 20 is in its unperturbed state. FIG. 19B shows the sensor 20 undergoing the application of a specified force provided by a user, applying the sensor 20 to the skin 15 of the user. FIG. 20 shows how the sensor 20 is encapsulated within the device 110.

The invention consists of small elastically deformable protrusions with a living hinge at the base of the protrusions all of which are part of the housing assembly stack and are molded as radial, outwardly angled, protrusions from the housing's lower seal that resides immediately around the perimeter of the microneedle array. These radial, outwardly angled protrusions are slightly longer than the microneedles and just long enough make contact with the skin when the sensor is pressed into the skin immediately before the tips of the microneedles. Due to the outward angle and elasticity of the protrusions, as these radial protrusions apply pressure to the skin they stretch the skin in the small area immediately around the microneedle array only where skin stretching is necessary for effective insertion and do not stretch any skin outside of this contained region. These small molded protrusions are pressured outward while the sensor is pressured downward and stretching is occurring and as they rotate and deform outward on the living hinge at the base they fall into a cavity that is also molded into the seal and connected to the base of the protrusions and living hinge. Once the protrusions are fully in the cavity, the bottom surface of the housing is level (flush) and does not interfere with microneedle insertion. Without these cavities, the protrusions would continue to apply pressure to the skin and potentially pull the microneedles out of the skin after insertion.

Additively, the living hinge is designed in a way that is applies pressure on the protrusions from the attachment point to keep the protrusions in the cavity. This is achieved with an arc shaped living hinge that adds a camming force as the protrusion travels from the extended position to the retracted position effectively holding the protrusion in the extended, or retracted position naturally and now allowing the protrusion to rest in any position between the extended and retracted position.

One embodiment is micro-stretching protrusions could be molded as separate part rather than one part with the seal or housing.

Another embodiment is micro-stretching protrusions could be rigid plastic rather that elastic and still actuate via living hinge.

Another embodiment is micro-stretching protrusions could be designed with a classic pivoting hinge rather than living hinge.

Another embodiment is micro protrusions with a texture on the tip designed to engage the skin with improved friction between the protrusions and skin.

Another embodiment is micro protrusions with adhesive on the skin-facing surfaces to engage the skin with improved friction/stiction.

Another embodiment is micro-protrusions actuated by a small spring. Another embodiment is various quantities of micro-protrusions 2, 3, 4, 5, 6, 7, +1, etc.

Another embodiment is micro-protrusions that are outwardly arc shaped and designed to roll on the skin as they rotate outward.

Another embodiment is micro-protrusions with small sharp tips on the end to assist is grabbing the skin for more effective stretching.

It is generally accepted in the microneedle development and application industry that inserting microneedles into the skin requires a prescribed minimum velocity at impact. Mechanically analogous to a nail gun, where the nail is accelerated into a piece of wood relying in inertial forces for effective insertion.

Applicators retain microneedles at some displacement distance away from the skin and then accelerate the microneedles into the skin at a rate fast enough to achieve insertion before the skin can elastically deform. This approach requires linear action slides or radial action pivots that typically increase the profile and surface area of the applicator. This approach to insertion also requires controlled input force to achieve proper impact velocity and has the unfortunate result of startling the subject (user) when the trigger is released and on impact.

The primary objectives of the present embodiments are: to lower the overall profile and surface area required to apply microneedles; To achieve consistent effective insertion with little or no displacement; To avoid startling sounds, slapping, and potential for pain to the user/wearer; To reduce the number of human factors and physics variables involved with the physics of insertion; Reduce the risk of off-perpendicular insertion; Reduce risk of microneedle shear (which can contribute to catastrophic brittle fracture); Reduce effects of small movements from the user; Reduce total impact energy required for insertion.

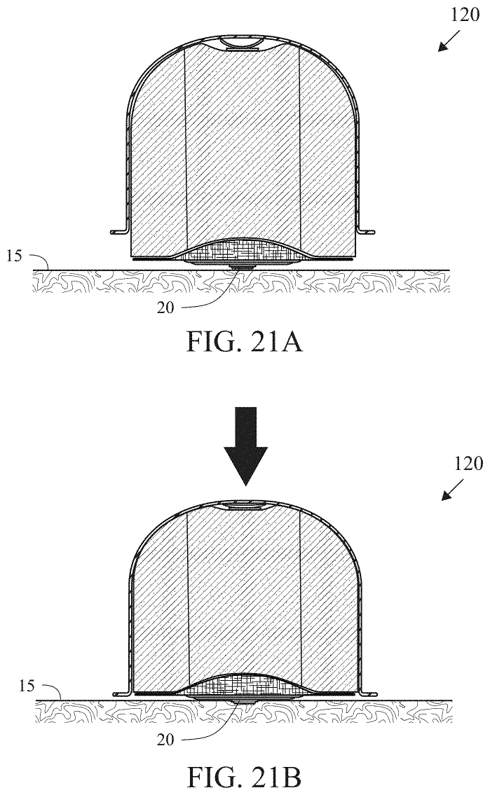

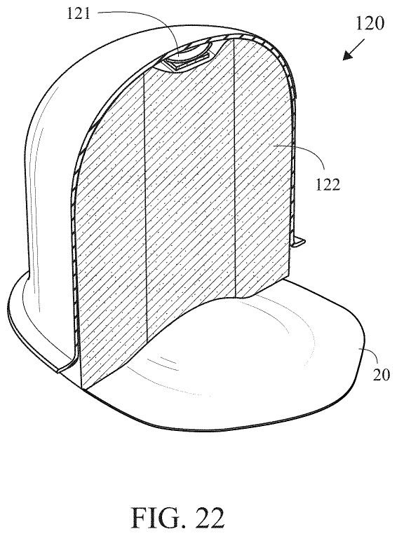

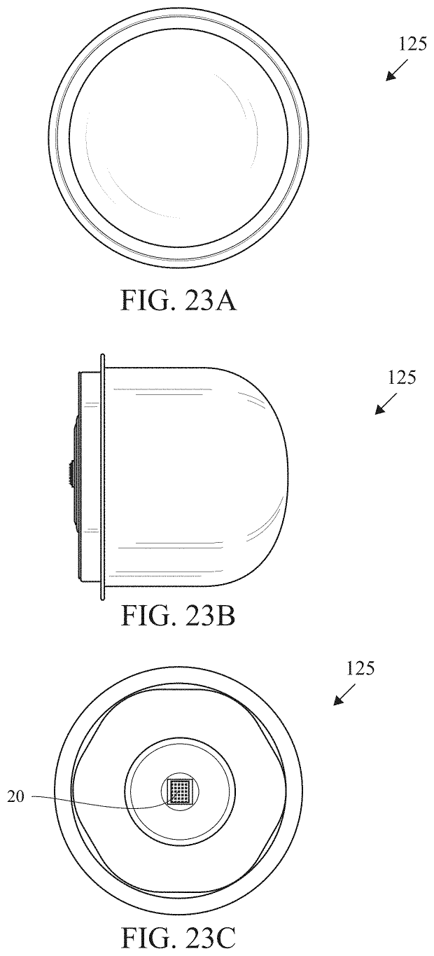

One aspect of the invention is a device 120 which consists of a mass 122 suspended a small distance above the microneedle array 20 and a metal spring dome 121 between the microneedle array 20 and the mass, as shown in FIGS. 21A-23C. The microneedle array is pressed by the user directly against the skin, touching the skin, with a downward force applied to the top of the mass until the metal dome 121 collapses (analogous action to a tact switch), as shown in FIG. 21A, accelerating the mass downward on a path to impact the back of the housing that holds the sensor array 20. The final effect being to “hammer” the microneedle array 20 into the dermis 15 in much the same way a hammer impact can insert a nail. In this invention, the needles are pressed against the skin before the hammering force is applied, as shown in FIG. 21A; this pre-loading of the needles onto the skin reduces the total energy required to insert the needles. Embodiments include: A compressed spring can be used to accelerate the mass rather than the press of a user's finger; Multiple impacts and multiple masses. Multiple impacts with one mass; A sensor housing designed to maximize the transfer of force from the top of the housing into the microneedle array; and increased or varying travel distances for the hammer.

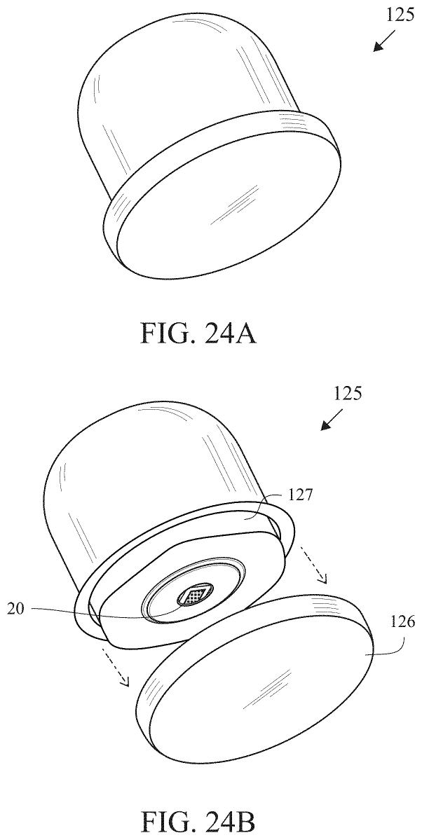

FIG. 24A-B shows a sterile barrier packaging 125 retaining a sensor 20 and featuring a user-removable protective cover 126, which is removed immediately prior to sensor application.

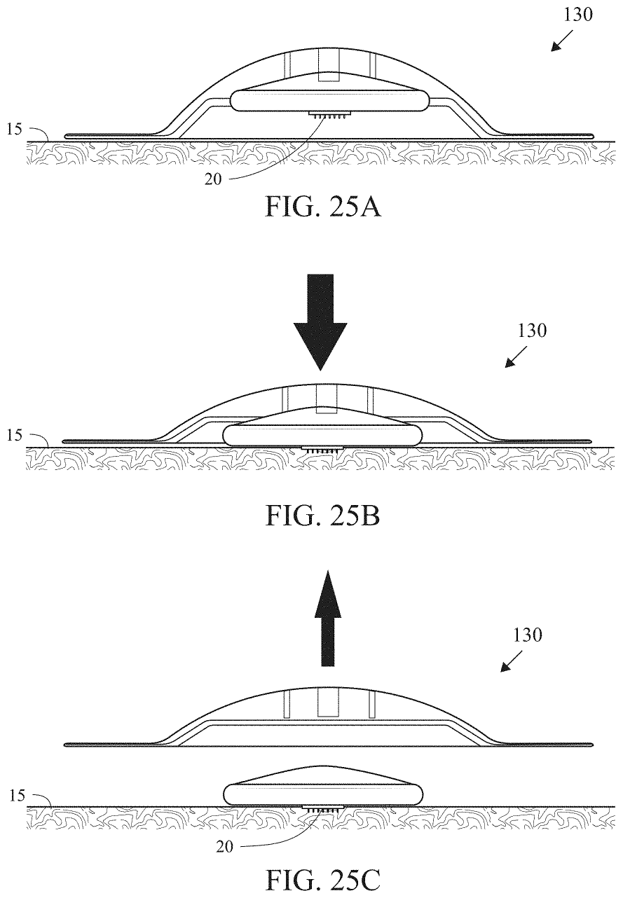

FIGS. 25A-C show a device 130 of an engineered failure application method featuring frangible elements integrated into the sterile barrier packaging. In FIG. 25A, the sensor 20 is in its unperturbed state. FIG. 25B shows the sensor 20 undergoing the application of a specified force provided by a user. FIG. 25C show the sensor 20 retained on the skin 15 of a user following the ‘press-on’/pressure application process applied by said user.

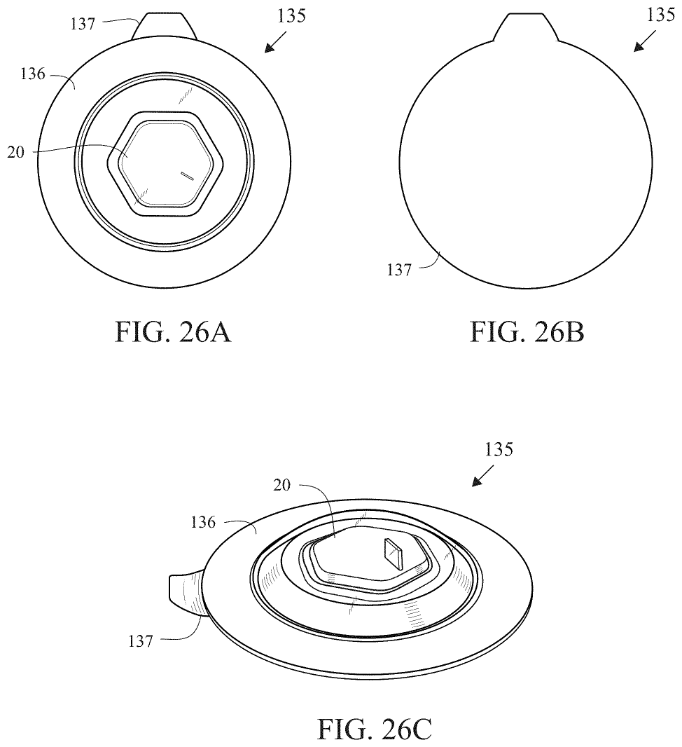

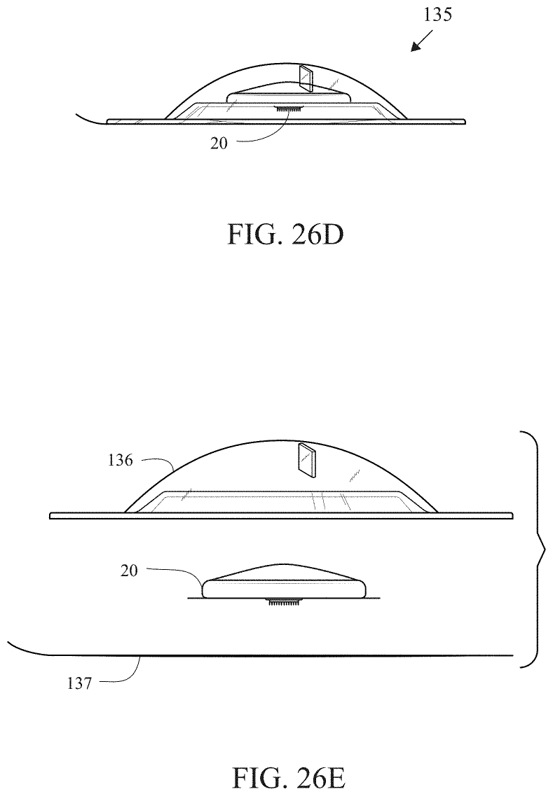

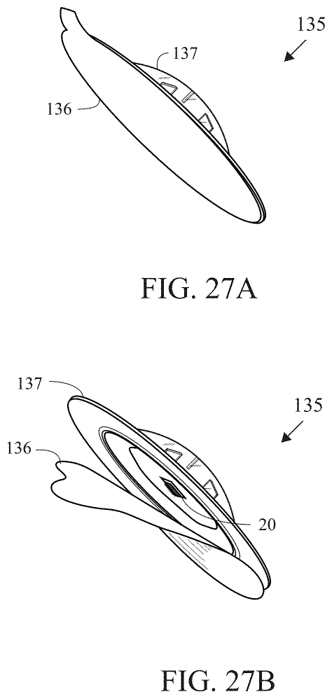

FIGS. 26A-27B show a sterile barrier packaging device 135 of an engineered failure application method. The device 135 is composed of a body 136 housing the sensor 20 and a peel-away cover 137. In certain embodiments, the removal of the protective cover 137 can expose a pressure-sensitive adhesive on the underside of the sterile barrier packaging, which is intended to adhere to the skin of a wearer and provide for stabilization of the sensor application process or the application of strain to the skin prior to sensor application.

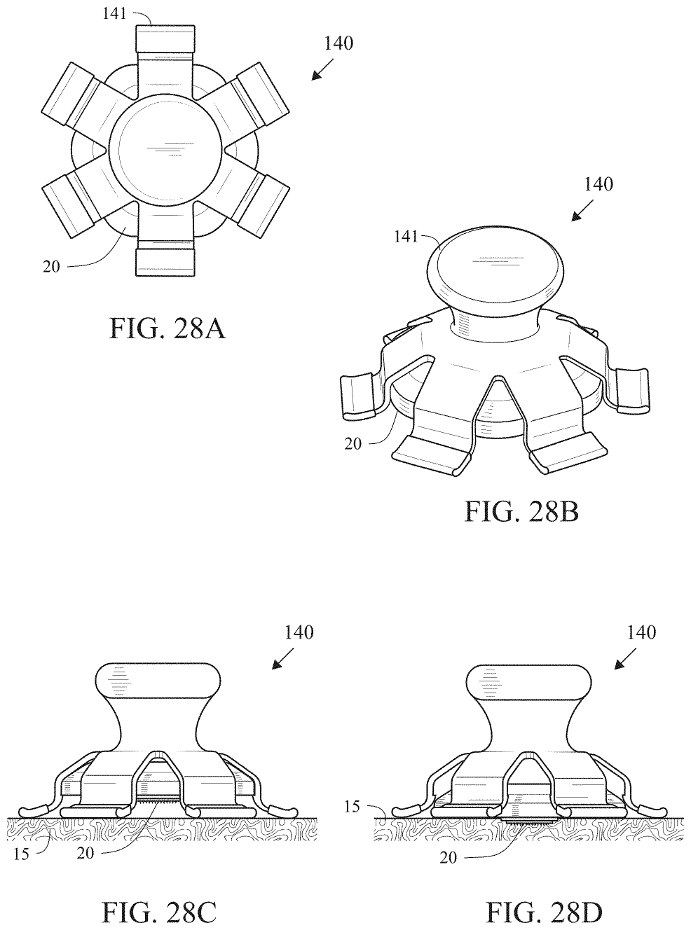

FIGS. 28A-D show a device 140 with a body 141 housing the sensor 20. FIG. 28D shows the sensor 20 after the application of a specified force provided by a user, applying the sensor 20 to the skin 15 of the user.

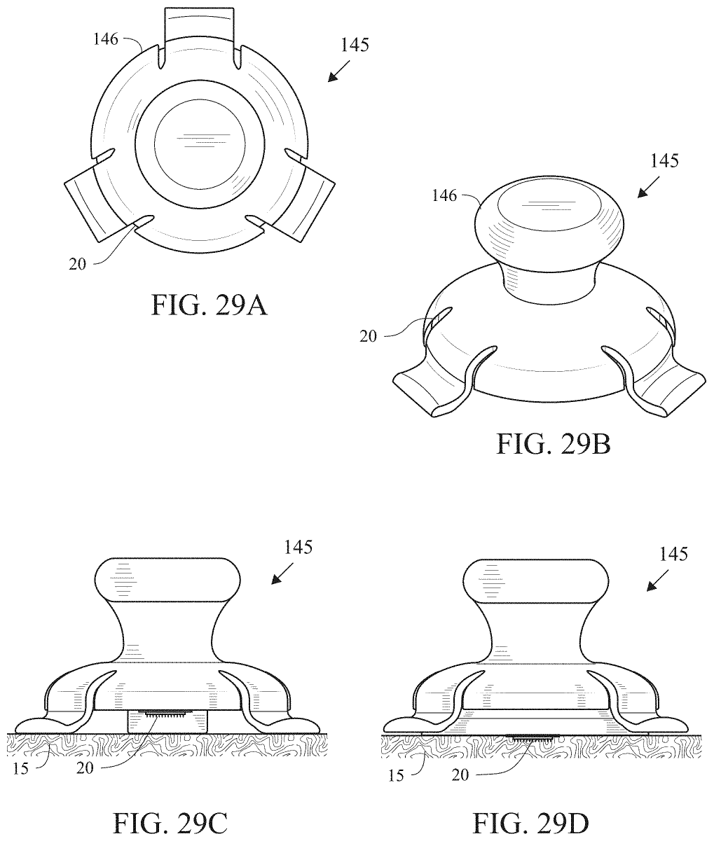

FIGS. 29A-D show a device 145 with a body 146 housing the sensor 20. FIG. 29D shows the sensor 20 after the application of a specified force provided by a user, applying the sensor 20 to the skin 15 of the user.

The analyte-selective sensor (SENSOR) is preferably a microneedle or microneedle array-based electrochemical, electrooptical, or fully electronic device configured to measure an endogenous or exogenous biochemical agent, metabolite, drug, pharmacologic, biological, or medicament in the dermal interstitium, indicative of a particular physiological or metabolic state in a physiological fluid of a user. Specifically, said microneedle array contains a plurality of microneedles, possessing vertical extent between 200 and 2000 μm, configured to selectively quantify the levels of at least one analyte located within the viable epidermis or dermis and in the vicinity of the papillary plexus, subpapillary plexus, or dermal plexus. Said microneedle array is contained and/or mounted to an enclosure or housing containing a power source, electronic measurement circuitry, a microprocessor, and a wireless transmitter. SENSOR is configured with a skin-facing adhesive (sensor adhesive) intended to adhere the said SENSOR for the desired wear duration.

The sensor retainer/carrier (CARRIER) secures the sensor in place and is responsible for accelerating the SENSOR during deployment towards the skin surface of a user.

A user clasps holder (HOLDER) with hand to position SENSOR over desired application area. The base of the holder includes flanges, which are configured to provide additional surfaces for the user to hold the applicator, resulting in increased control of placement on skin and during acceleration of the SENSOR during application.

A shaft/threaded insert (SHAFT) is a pivot axle for the CARRIER and point of attachment of said CARRIER to the HOLDER. It enables the CARRIER to undergo radial motion that follows an arc trajectory.

A torsion spring (SPRING) augments the acceleration of SENSOR once applicator is deployed by the conversion of stored potential energy to kinetic energy.

A ball nose/spring plunger (BALL) applies a prescribed interference to retain CARRIER in the “loaded” position (primed for deployment/application). Adjustments to the tension embodied by the BALL results in a concomitant adjustment to the trigger force of the applicator. Threading SENSOR further into HOLDER manifests increased interference and hence higher trigger force required to deploy SENSOR.

A rubber pad (PAD) imparts additional friction/traction to secure

HOLDER to desired location on the skin of a user and simultaneously decreases the probability of lateral movements during application of SENSOR.

A nylon tip (TIP) secures BALL in desired location; used in conjunction with a set screw.

A set screw (SCREW) secures BALL in desired location; used in conjunction with TIP.

An auto release (RELEASE) secures SENSOR during while the applicator is primed. The RELEASE is characterized by a prescribed degree of compliance/flexibility. In the primed position, BALL applies pressure to the auto-release causing it to deform in a manner which secures SENSOR in an immotile position. Once deployed, the RELEASE returns to its initial position/shape and SENSOR is released.

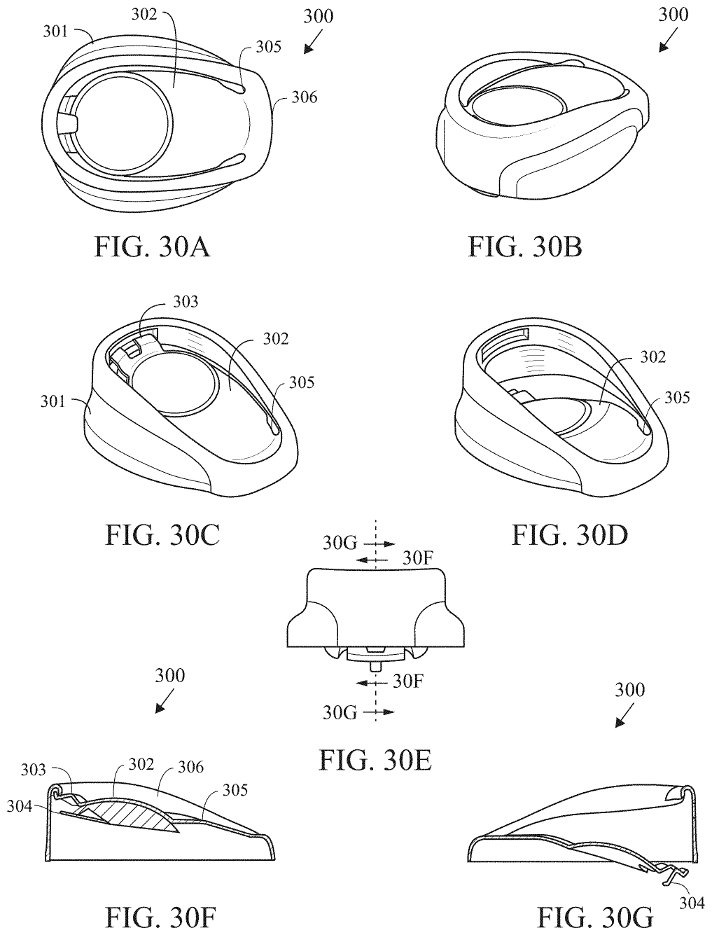

FIGS. 30A-30G show a spring assisted device 300 of the invention. The device 300 consists of a body 306 with a finger flange 301 for stability during application, tongue 302, locking tab 303, leg 304, and a spring assist leaf 305. The locking tab 303 controls the trigger release force. The leg 304 holds the sensor 20 when the tab 303 is compressed, and auto-releases the sensor 20 when the tab 303 is decompressed. FIGS. 30C and 30F shows the tongue 302 in a loaded position, ready to be triggered. FIGS. 30D and 30G show the tongue in a relaxed, unloaded position, after the sensor is released.

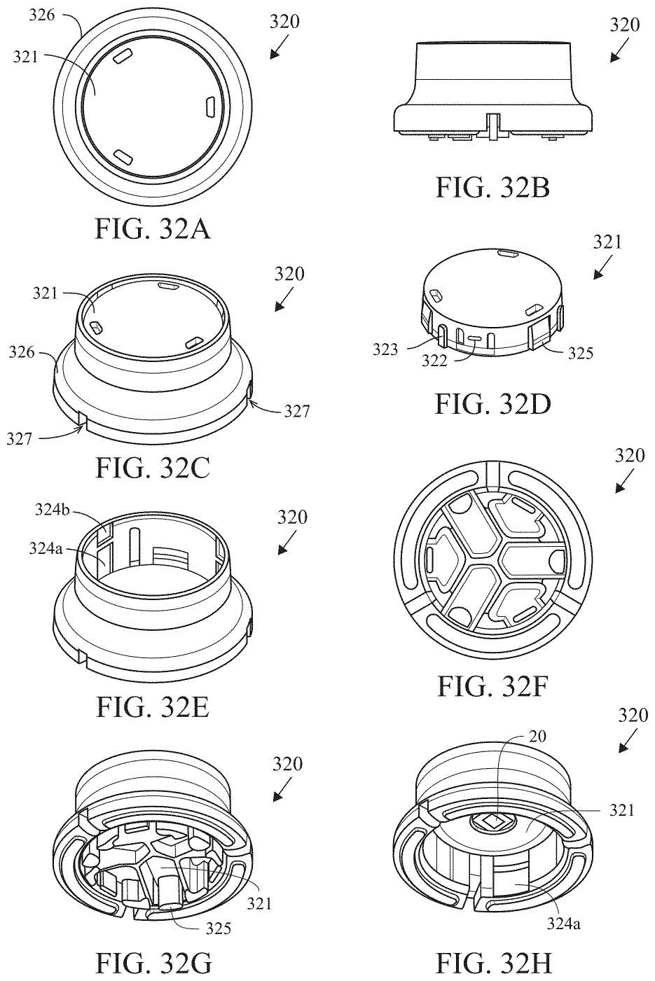

FIGS. 32A-H shows a two-piece applicator device 320, consisting of a frame 326 and a piston 321, configured with an auto-release. The piston 321 is shown in FIG. 32D. The piston consists of alignment rails 323, release tabs 322, and auto-release leg 325. FIG. 32E shows an upper cavity 324b for release tabs, and a lower release tab cavity 324a, and air vents 327. The release tabs 322 control trigger force. The auto-release leg 325 holds and releases the piston. FIGS. 32C and 32H show the piston 321 in a loaded position, holding the sensor 20. The auto-release leg 325 prevents movement in the loaded position. FIGS. 32E-G show the piston 321 after it was triggered. FIG. 32G shows the auto release leg 325 is relaxed into the cavity 324 in the unloaded position.

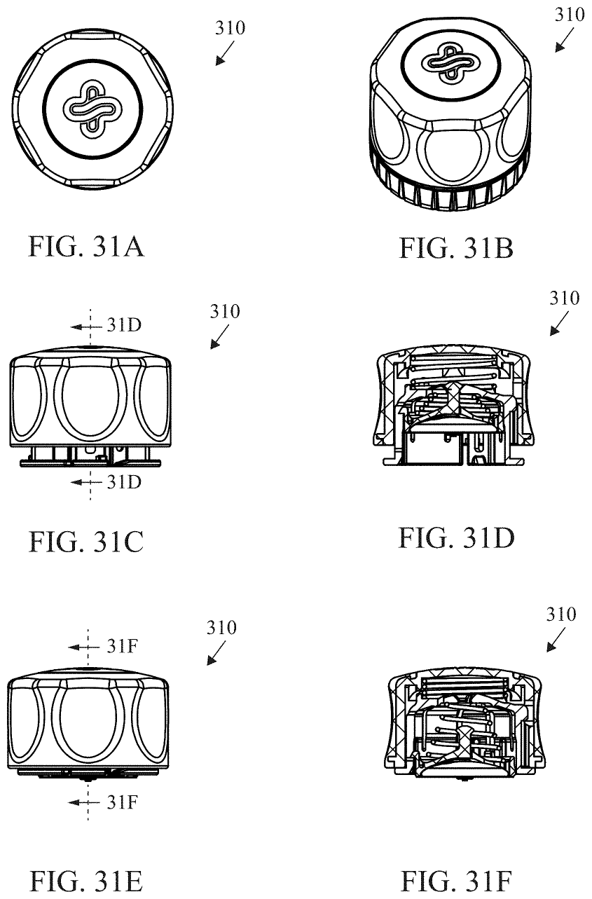

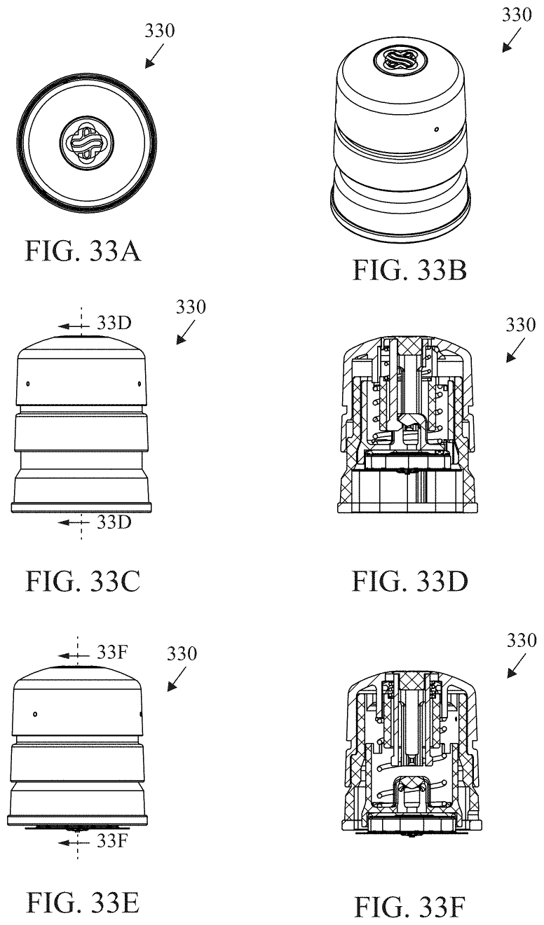

FIGS. 31A-F and FIGS. 33A-F show another embodiment of the invention as devices 310 and 330, respectively. In FIGS. 31C-D and FIGS. 33C-D, the devices 310 and 330 are shown in a cocked position. FIGS. 31E-F and FIGS. 33E-F are shown in a deployed position.

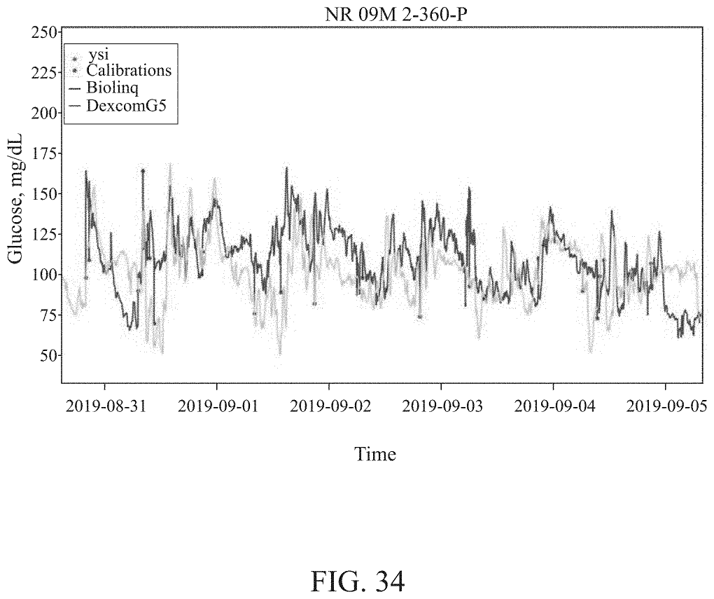

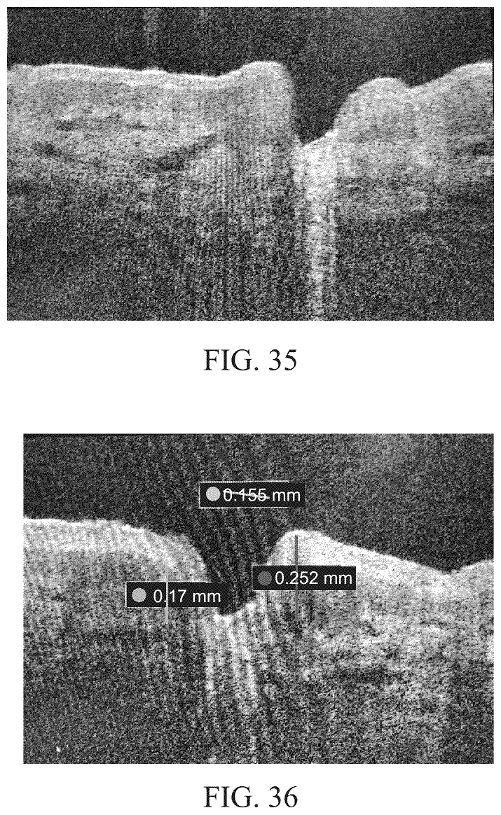

FIG. 34 is a time-series dataset substantiating the ability of a microneedle array-based glucose-selective sensor to be applied without a mechanical application mechanism to track glucose in the dermis of a wearer in a quantitative fashion. Correlate glucose measures (Dexcom® G5™ CGM, YSI® Model 2300 Electrochemical Analyzer™) as well as periodic calibrations with fingerstick blood samples (Bayer® Contour NEXT™) is provided in the plot.

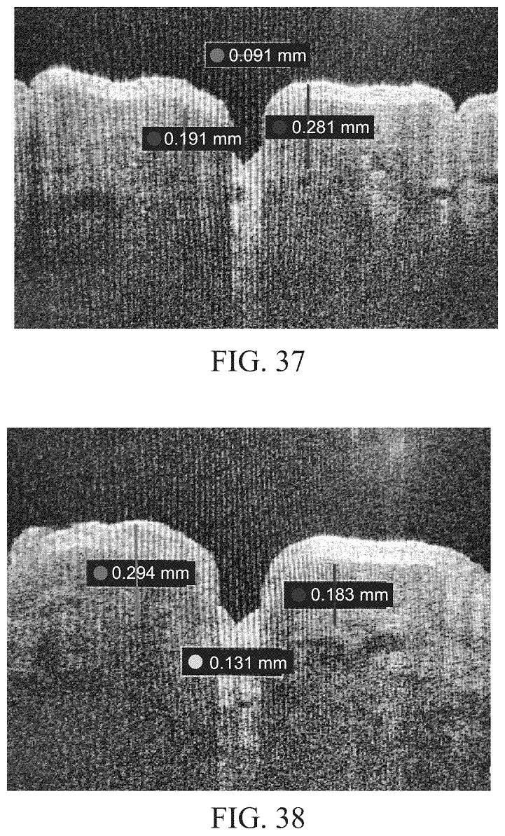

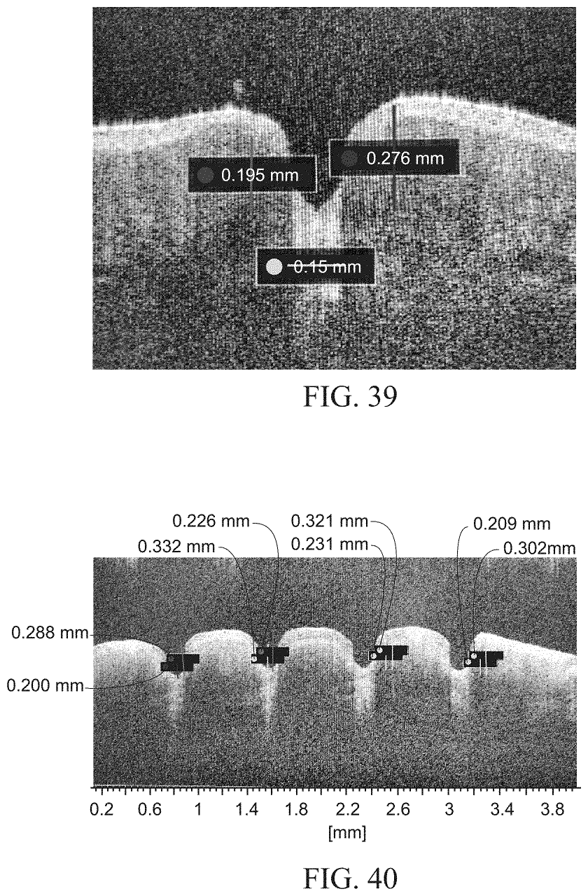

FIGS. 35-40 are images from an optical coherence tomography (OCT) analysis. FIG. 35 is of an array with seven microneedles with strain applied to the skin during application. FIG. 36 is an OCT analysis of an array with seven microneedles with the skin in a native state (no external strain imposed). FIG. 37 is an OCT analysis of an array with thirty-seven microneedles with the skin in a native state (no external strain imposed). FIG. 38 is an OCT analysis of an array with thirty-seven microneedles with the skin in a native state (no external strain imposed). FIG. 39 is an OCT analysis of an array with thirty-seven microneedles with strain applied to the skin during application. FIG. 40 is an OCT analysis of an array with thirty-seven microneedles applied with a mechanical applicator with strain applied to the skin during application.

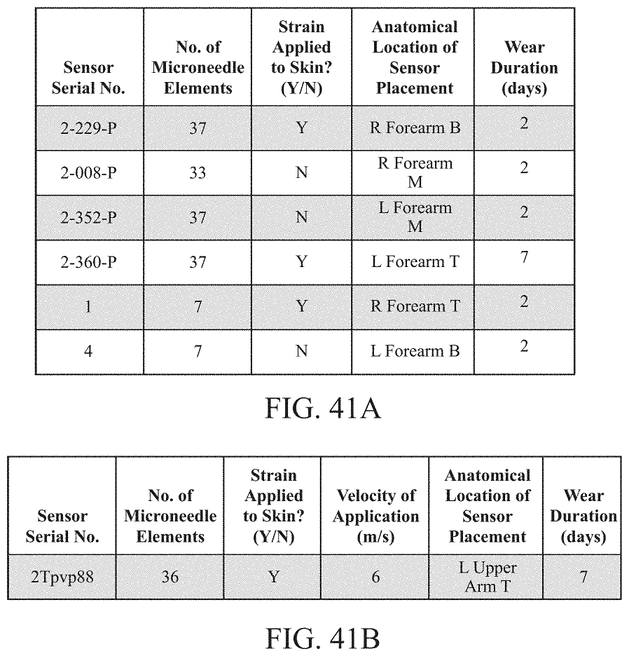

FIG. 41A shows data plots of experimental configurations of microneedle array analyte-selective sensors applied to a wearer without the aid of a mechanical applicator mechanism. FIG. 41B shows control configurations of a microneedle array analyte-selective sensors applied to a wearer with the aid of a mechanical applicator mechanism.

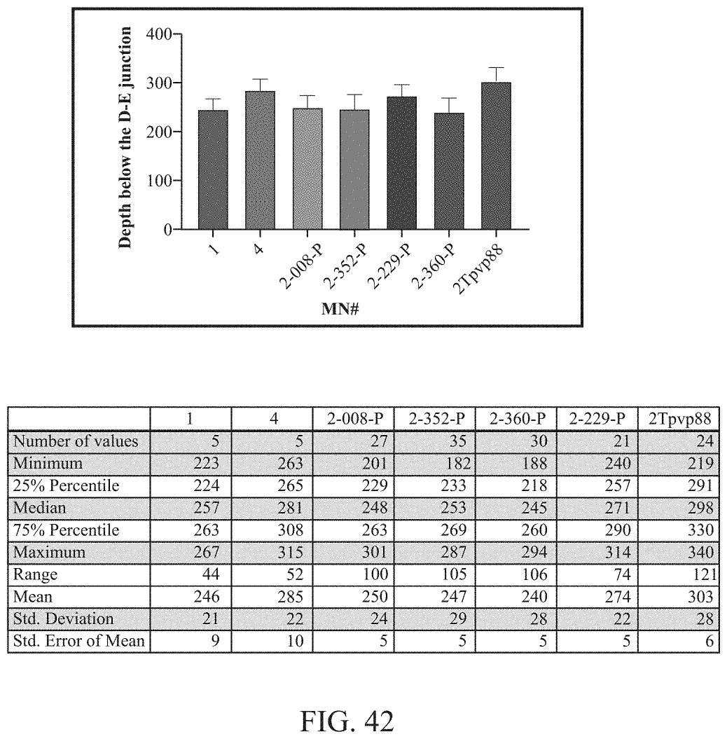

FIG. 42 is a representative bar chart and descriptive statistics of the insertion depth below the skin surface embodied by each microneedle array analyte-selective sensor configuration studied.

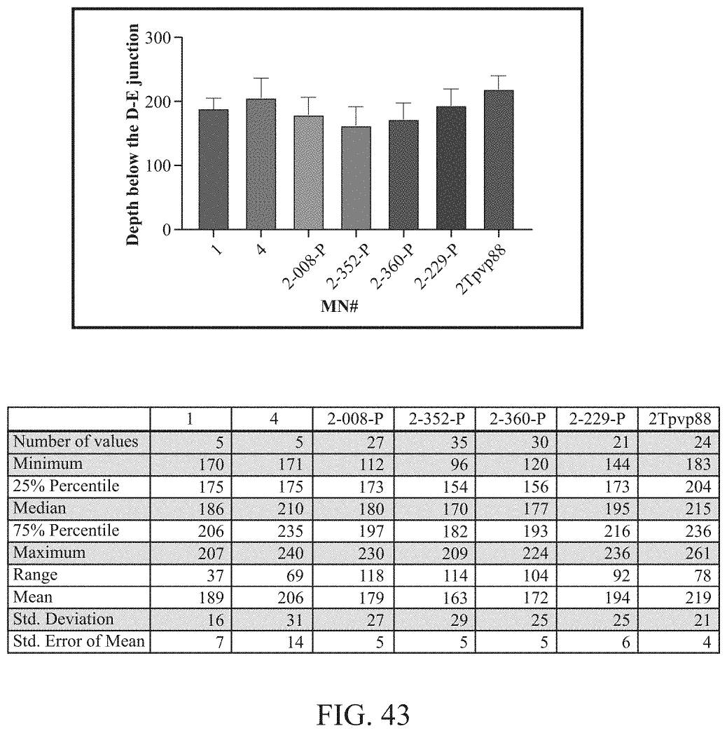

FIG. 43 is a representative bar chart and descriptive statistics of the insertion depth below the epidermal-dermal junction embodied by each microneedle array analyte-selective sensor configuration studied.

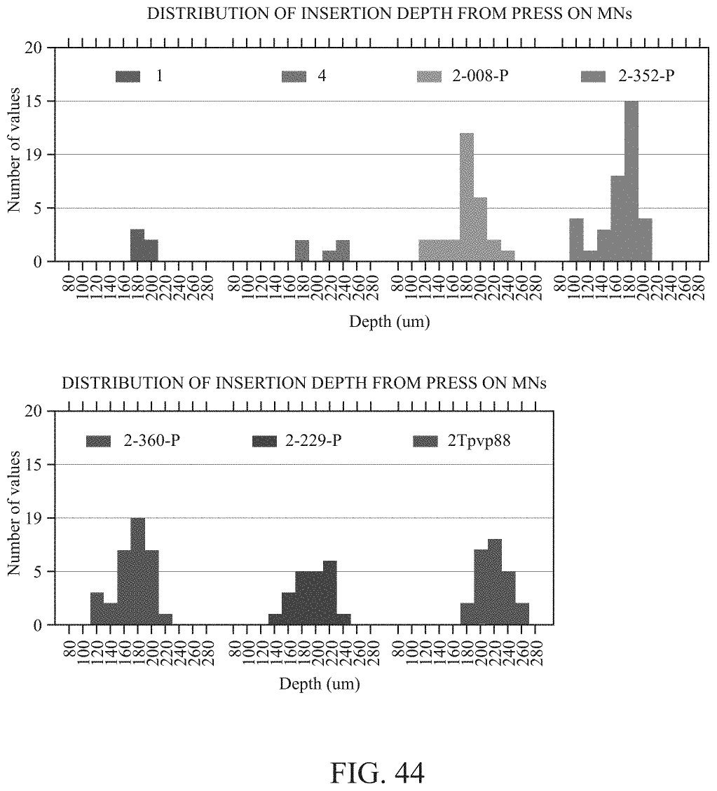

FIG. 44 is a histogram illustrating the distribution of insertion depth embodied by each microneedle array analyte-selective sensor configuration studied.

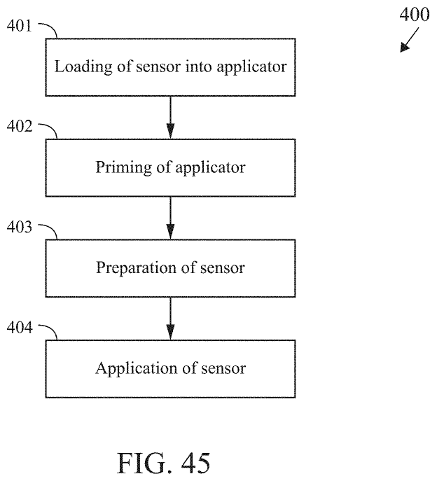

A method 400 of practicing the invention is shown in FIG. 45 and begins with loading the SENSOR into the applicator, as in step 401. The SENSOR is inserted into a cavity within CARRIER and is thereby retained within CARRIER. For the sake of clarity, CARRIER is not engaged with BALL (i.e. the applicator is not primed). Furthermore, the RELEASE component is not engaged by BALL and remains in its unloaded state, which results in loose engagement between RELEASE and SENSOR. When RELEASE component is not deformed by BALL, SENSOR can be easily inserted and removed from CARRIER RELEASE is comprised of at least one of an elastomer, thermoplastic, metal, or elastically-deformable material when subject to mechanical stress.

Next, step 402, includes priming of the applicator, as shown in FIG. 11. The CARRIER is rotated upward until BALL engages a mating feature in CARRIER, thereby rendering said CARRIER immobile. The mating feature on CARRIER possesses inverted geometry to BALL and secures CARRIER in a fixed position at a specified location. Said mating feature on CARRIER is concentric with a cylindrical feature on RELEASE. The cylindrical feature on RELEASE comprises a shaft, which is free to engage in linear motion within a cylindrical bore on CARRIER Upon engagement of BALL with CARRIER, the cylindrical shaft on RELEASE is deformed by BALL, thereby causing RELEASE to deform in the direction of SENSOR, as shown in FIG. 11A. Upon deformation of release by BALL, said RELEASE engages SENSOR, compressing SENSOR against features within CARRIER, retaining the SENSOR in a fixed position. SENSOR is stabilized by a plurality of vertical bosses that retain said SENSOR in an immobile position (i.e. free from rotational and translational motion) in the x-y plane; motion in z-axis is uninhibited. RELEASE engages SENSOR by a friction force given by interference fit with SENSOR. This provides sufficient force to retain SENSOR in the z-axis when CARRIER is in the loaded position and during the removal of the adhesive liner.

Next, step 403, includes preparing the SENSOR. With RELEASE engaging/securing SENSOR, user removes adhesive liner from skin-facing surface of said sensor. The applicator is then placed over the desired application site. Flanges on the exterior of HOLDER, in combination with PAD located on the skin-facing surface of the applicator allow for securement of the applicator in the desired location in all three cardinal axes. This feature is necessary due to the stored potential energy in SPRING, which, upon deployment, causes rapid acceleration of HEAD towards the skin of a user. This rapid acceleration may give rise to recoil in the HOLDER that could destabilize the system.

Next, step 404, includes application of the SENSOR. The user activates applicator by depressing CARRIER until required minimum actuation force is achieved, desirably between 0.3 and 30 Newtons. Once the minimum actuation force is exceeded, BALL releases CARRIER The actuation force can be increased or decreased, as desired, by adjusting the amount of engagement of BALL. The application force and velocity is directly related to the actuation force and the strength of SPRING. The sensor is accelerated via SPRING and applied force until it impacts the skin of the user, thereby applying SENSOR. The application force and velocity can be modulated by an appropriate selection of SPRING stiffness/constant. SPRING augments the impact velocity via conversion of stored potential energy to kinetic energy. Furthermore, SPRING improves the consistency of the final impact velocity to compensate for variability in user-applied force to deploy SENSOR. Once CARRIER is deployed, BALL no longer deforms RELEASE and SENSOR is released from CARRIER as interference fit is no longer applied. Under current embodiments, RELEASE exerts loose coupling to SENSOR, even when released, to help stabilize SENSOR AND CARRIER through the acceleration, impact, and application. Following this process, SENSOR is applied to the skin of the user and the applicator can be removed. The securement force of SENSOR to CARRIER is significantly less than the securement force of SENSOR ADHESIVE to skin. This ensures that SENSOR can be easily released from the applicator once applied. In alternative embodiments, applicator is configured to function without SPRING. In the absence of SPRING, the force required by the user is increased to around 30 N to achieve a target velocity of approximately 5 m/s, where with SPRING the same velocity can be achieved with less than a 20 N force applied by the user. These figures depend on the overall mass of SENSOR and CARRIER, constant of SPRING, length of CARRIER, and potentially other variables.

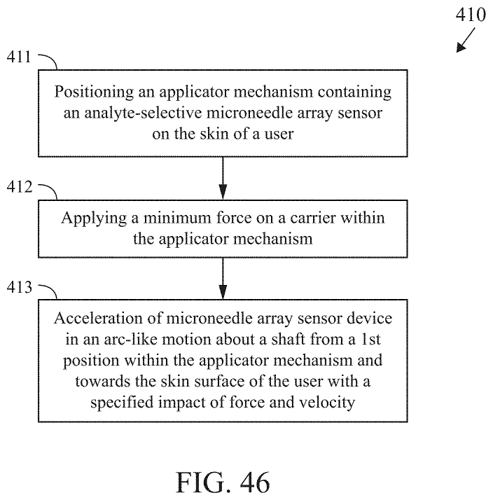

Another method 410 of the invention is shown in FIG. 46. Step 411 includes positioning an applicator mechanism containing a sensor on the skin of a user. This positioning indicates the future location of the sensor placement. Step 412 includes applying a minimum force on a carrier within the applicator mechanism. This force causes the spring plunger to retract and a deformed release mechanism to return to its native shape. Finally, step 413 is the acceleration of the sensor device in an arc-like motion about a shaft from a first position within the applicator mechanism and towards the skin surface of a user with a specified impact force and velocity. The insertion depth beneath the skin surface of a user is dependent on the velocity and mass (momentum) of the microneedle array when it impacts the skin.

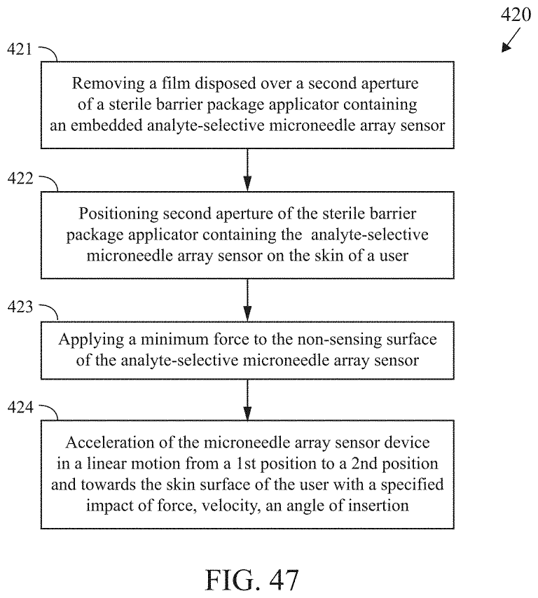

Another method 420, as shown in FIG. 47, is for the insertion of an analyte-selective microneedle array sensor into a dermal stratum of a user by means of a sterile barrier package applicator containing a first aperture, second aperture, and body portion. This method 420 begins with step 421, removing a film disposed over a second aperture of a sterile barrier package applicator containing an embedded sensor. Removal of the film exposes sensor surface of the analyte-selective microneedle array sensor.

Next, step 422, is positioning second aperture of the sterile barrier package applicator, containing the sensor, on the skin of a user. Positioning an applicator mechanism indicates future location of placement of the sensor.

Next, step 423, is applying a minimum force to the non-sensing surface of the sensor. Applying a minimum force compromises an engineering fit retaining the sensor to the body portion.

Next, step 424, is the acceleration of the sensor device in a linear motion from a first position to a second position and towards the skin surface of a user with a specified impact force, velocity, and angle of insertion. The insertion depth beneath the skin surface of a user is dependent on the velocity and mass (momentum) of the microneedle array when it impacts the skin.

The inputs of the invention include a user-directed application of force to the CARRIER. Said application of force, of a minimum specified magnitude, is intended to deploy CARRIER and accelerate SENSOR to the skin of a user at a prescribed velocity and impact force.

The outputs of the invention include application of SENSOR to the skin of a user. A SENSOR applied to the skin surface of a user and retained in the desired position by means of a skin-facing adhesive. Said application process results in the microneedle constituents of said SENSOR penetrating the stratum corneum and accessing the interstitial fluid of the viable epidermis, papillary dermis, or reticular dermis in order to impart the sensing operation of at least one of a circulating endogenous or exogenous biochemical agent, metabolite, drug, pharmacologic, biological, or medicament.

McCanna et al., U.S. Pat. No. 9,933,387 for a Miniaturized Sub-Nanoampere Sensitivity Low-Noise Potentiostat System is hereby incorporated by reference in its entirety.

Windmiller, U.S. patent application Ser. No. 15/177,289, filed on Jun. 8, 2016, for a Methods And Apparatus For Interfacing A Microneedle-Based Electrochemical Biosensor With An External Wireless Readout Device is hereby incorporated by reference in its entirety.

Wang et al., U.S. Patent Publication Number 20140336487 for a Microneedle Arrays For Biosensing And Drug Delivery is hereby incorporated by reference in its entirety.

Windmiller, U.S. patent Ser. No. 10/092,207 for a Tissue Penetrating Electrochemical Sensor Featuring A Co Electrodeposited Thin Film Comprised Of A Polymer And Bio-Recognition Element is hereby incorporated by reference in its entirety.

Windmiller, et al., U.S. patent application Ser. No. 15/913,709, filed on Mar. 6, 2018, for Methods For Achieving An Isolated Electrical Interface Between An Anterior Surface Of A Microneedle Structure And A Posterior Surface Of A Support Structure is hereby incorporated by reference in its entirety.

PCT Publication Number WO2018071265 for an Electro-Deposited Conducting Polymers For The Realization Of Solid-State Reference Electrodes For Use In Intracutaneous And Subcutaneous Analyte-selective Sensors is hereby incorporated by reference in its entirety.

Windmiller et al., U.S. patent application Ser. No. 15/961,793, filed on Apr. 24, 2018, for Heterogeneous Integration Of Silicon-Fabricated Solid Microneedle Sensors And CMOS Circuitry is hereby incorporated by reference in its entirety.

Windmiller et al., U.S. patent application Ser. No. 16/051,398, filed on Jul. 13, 2018, for Method And System For Confirmation Of Microneedle-Based Analyte-Selective Sensor Insertion Into Viable Tissue Via Electrical Interrogation is hereby incorporated by reference in its entirety.

Windmiller et al., U.S. patent application Ser. No. 16/701,784, filed on Dec. 3, 2019, for Devices And Methods For The Generation Of Alerts Due To Rising Levels Of Circulating Ketone Bodies In Physiological Fluids is hereby incorporated by reference in its entirety.

Windmiller et al., U.S. patent application Ser. No. 16/824,700, filed on Mar. 20, 2020, for Devices and Methods For The Incorporation Of A Microneedle Array Analyte-Selective Sensor Into An Infusion Set, Patch Pump, Or Automated Therapeutic Delivery System is hereby incorporated by reference in its entirety.

Windmiller et al., U.S. patent application Ser. No. 16/899,541, filed on Jun. 11, 2020, for a Mechanical Coupling Of An Analyte-Selective Sensor And An Infusion System And Information Conveyance Between The Same is hereby incorporated by reference in its entirety.

From the foregoing it is believed that those skilled in the pertinent art will recognize the meritorious advancement of this invention and will readily understand that while the present invention has been described in association with a preferred embodiment thereof, and other embodiments illustrated in the accompanying drawings, numerous changes modification and substitutions of equivalents may be made therein without departing from the spirit and scope of this invention which is intended to be unlimited by the foregoing except as may appear in the following appended claim. Therefore, the embodiments of the invention in which an exclusive property or privilege is claimed are defined in the following appended claims.

We claim as our invention the following:

1. An applicator device configured for the insertion of an analyte-selective microneedle array sensor into a dermal stratum of a user, said device comprising:
a body portion configured to be grasped with a hand of said user;
a carrier configured to retain said sensor and accelerate sensor during deployment towards the skin surface of said user;
a pivoting member at a proximal end of said carrier configured to enable carrier to undergo radial motion about said pivoting member;
a catch-release mechanism configured to apply an engineering fit to retain carrier in a first position; and
a release mechanism configured to deform its shape upon compression by said catch-release mechanism;
wherein a user-directed application of a specified force to the carrier causes the catch-release mechanism to retract and the release mechanism to return to its native shape, thereby to effect the acceleration of the microneedle array sensor device in an arc-like motion about said pivoting member and towards the skin surface of a user with a specified impact force and impact velocity.

2. The device of claim 1, wherein said microneedle array sensor is an electrochemical, electrooptical, or fully electronic device.

3. The device of claim 1, wherein said microneedle array sensor is configured to measure at least one of an endogenous or exogenous biochemical agent, metabolite, drug, pharmacologic, biological, or medicament indicative of a particular physiological or metabolic state in a physiological fluid of a user.

4. The device of claim 1, wherein said microneedle array sensor contains a housing containing a power source, electronic measurement circuitry, a microprocessor, and a wireless transmitter.

5. The device of claim 1, wherein said carrier is configured to retain said microneedle array sensor by means of at least one of an interference fit, friction fit, press fit, clearance fit, location fit, and a magnetic retainer.

6. The device of claim 1, wherein said pivoting member is at least one of a hinge, shaft, tongue, and an elastically-deformable membrane.

7. The device of claim 1, wherein said catch-release mechanism is a spring plunger.

8. The device of claim 1, wherein said first position is recessed within the body portion.

9. The device of claim 1, wherein said user-directed application of a specified force is mediated with the press of a finger of said user.

10. The device of claim 1, wherein said impact force is between 0.3 N and 30 N.

11. The device of claim 1, wherein said impact velocity is between 0.15 m/s and 15 m/s.

12. A sterile barrier package applicator device, said sterile barrier package applicator device comprising:
a first aperture;
a second aperture;
a body portion;
an analyte-selective microneedle array sensor retained by an engineering fit in a first position within said body portion, the non-sensing surface of said analyte-selective microneedle array positioned in proximity to said first aperture;
a film disposed over said second aperture of said sterile barrier package, said film configured to be removed by a user;
wherein a user-directed application of a minimum force to the non-sensing surface of said analyte-selective microneedle array compromises said engineering fit, thereby to effect the acceleration of the microneedle array sensor device in a linear motion from a first position to a second position and towards the skin surface of a user with a specified impact force, impact velocity, and angle of insertion.

13. The device of claim 12, wherein said first aperture, second aperture, and body portion comprise a singular actuation element.

14. The device of claim 13, wherein said singular actuation element comprises a pivoting member.

15. The device of claim 14, wherein said pivoting member is at least one of a hinge, shaft, tongue, and an elastically-deformable membrane.

16. A method for the insertion of an analyte-selective microneedle array sensor into a dermal stratum of a user by means of a sterile barrier package applicator containing a first aperture, second aperture, and body portion, said method comprising:
removing a film disposed over said second aperture of said sterile barrier package applicator;
positioning second aperture of said sterile barrier package applicator containing said analyte-selective sensor on the skin of a user;
applying a minimum force to the non-sensing surface of said analyte-selective microneedle array sensor;
wherein the application of a minimum force by a user compromises an engineering fit retaining said analyte-selective microneedle array sensor to said body portion, thereby to effect the acceleration of the microneedle array sensor device in a linear motion from a first position to a second position and towards the skin surface of a user with a specified impact force, impact velocity, and angle of insertion.

17. The microneedle array sensor of claim 16, wherein said microneedle array sensor contains a housing containing a power source, electronic measurement circuitry, a microprocessor, and a wireless transmitter, and wherein said housing is configured to control at least one of deployment initiation, angle of application, impact force, velocity, and tautness of the skin of a user, and wherein said housing is configured to automatically decouple from said microneedle array sensor upon a user-directed application of a minimum force with no secondary action required from said user.

18. The microneedle array sensor of claim 16, wherein said microneedle array sensor is configured with a skin-facing adhesive intended to adhere the said sensor to the skin surface of said wearer for an intended wear duration.

19. The microneedle array sensor of claim 16, wherein said skin-facing adhesive contains an adhesive liner that is coupled to said film, and wherein the removal of said film by a user causes the concomitant removal of said coupled adhesive liner.

20. The method of claim 16, wherein said minimum force is between 0.3 N and 30 N.

21. The method of claim 16, wherein said linear motion from a first position to a second position causes the presentation of at least one of a tactile feedback and an audible feedback to said user.

22. The method of claim 16, wherein said impact force is between 0.3 N and 30 N and said impact velocity is between 0.15 m/s and 15 m/s.

23. The method of claim 16, wherein the difference between said first position and said second position defines a travel distance.

24. The method of claim 23, wherein said minimum force and travel distance defines the insertion velocity.

25. The method of claim 16, wherein the act of positioning second aperture on the skin of a user applies tension to said skin.

26. The method of claim 17, wherein said housing contains a feature to reduce the motion of the skin surface, thereby reducing deflection of said skin during application of said microneedle array sensor.

27. The method of claim 16, wherein the said positioning of the second aperture of the sterile barrier package applicator on the skin of a user causes a tensile strain directed radially from the center of the said second aperture on skin of said user.

28. The method of claim 16, wherein said application of a minimum force by a user is assisted by means of a kinetic energy storage element.

29. An applicator device configured for the insertion of an analyte-selective microneedle array sensor into a dermal stratum of a user, said device comprising:
a body portion configured to be grasped with a hand of said user;
a recessed actuation portion configured to be pressed with a finger of said user;
a carrier configured to retain said sensor and accelerate sensor during deployment towards the skin surface of said user;
a gating feature configured to prevent carrier movement until a minimum force is applied; and
a disengagement feature configured to release the sensor upon deployment;
wherein a user-directed application of a specified force to the actuation area causes the carrier to overcome the gating feature, thereby to effect the acceleration of the microneedle array sensor device towards the skin surface of a user with a specified impact force and impact velocity.

30. The device of claim 29, wherein user-directed application of a specified force is assisted by means of a kinetic energy storage element.
